(19) United States
(12) Patent Application Publication
(10) Pub. No.: US 2004/0170977 A1
(43) Pub. Date: Sep. 2, 2004

(54) Epigenetic sequences for esophageal adenocarcinoma

(76) Inventor: Peter Laird, Prospect Street, CA (US)

Correspondence Address: Barry L Davison, Davis Wright Tremaine, 2600 Century Square, 1501 Fourth Avenue, Seattle, WA, 98101-1688, US

(21) Appl. No.: 10/240,126

(22) PCT Filed: Apr. 2, 2001
(86) PCT No.: PCT/US01/10658

(30) Foreign Application Priority Data
Mar. 31, 2000 (US) 60193839

(51) Int. Cl. C12Q 1/68

(52) U.S. Cl. 435/6

(57) ABSTRACT

There is disclosed a diagnostic or prognostic assay for cancer, particularly gastrointestinal and esophageal adenocarcinoma. Specifically, the present invention provides a methylation pattern that can be assayed by standard methylation assays of CpG islands, including which genes are hypermethylated and which genes are unmethylated in gastrointestinal and esophageal adenocarcinomas, Barrett's esophagous, and normal squamous mucosa.

### CROSS-REFERENCE TO RELATED APPLICATIONS

This application claims priority to U.S. Provisional Patent Application Serial No. 06/193,839, entitled EPIGENETIC SEQUENCES FOR ESOPHAGEAL ADENOCARCINOMA, filed 31 Mar. 2000.

### STATEMENT REGARDING FEDERALLY-SPONSORED RESEARCH

This work was supported by NIH/NCI grant R01 CA 75090 to P.W.L. The United States has certain rights in this invention, pursuant to 35 U.S.C. § 202(c)(6).

### TECHNICAL FIELD OF THE INVENTION

The present invention provides a diagnostic or prognostic assay for gastrointestinal adenocarcinoma, and particularly esophageal adenocarcinoma (“EAC”). Specifically, the present invention provides a multi-geneic epigenetic fingerprint or methylation pattern, that can be assayed by standard methylation assays of CpG island methylation status, and that comprises the relative methylation status of two or more genes in gastrointestinal carcinomas, normal squamous cells, and EAC.

### BACKGROUND OF THE INVENTION

DNA methylation and cancer. DNA methylation patterns are frequently altered in human cancers. These methylation changes include genome-wide hypomethylation as well as regional hypermethylation (Jones & Laird, Nat Genet. 21:163-167, 1999). Aberrant hypermethylation in cancer cells often occurs at CpG islands, which are generally protected from methylation in normal tissues. Hypermethylation of promoter CpG islands (that is, CpG islands located in promoter regions of genes) has been associated with transcriptional silencing in many types of human cancers.

Methylation patterns of genes can provide different types of useful information about a cancer cell. First, each tumor type (i.e., breast, colon, esophagus, etc.) has a characteristic set of genes with an increased propensity to become methylated (Costello et al., Nat. Genet. 24:132-138, 2000). For example, RB1 is known to be hypermethylated in retinoblastoma (Stirzaker et al., Cancer Res. 57:2229-2237, 1997; Sakai et al., Am. J. Hum. Genet. 48:880-888, 1991), but not in acute myelogenous leukemia (Kornblau & Qiu, Leuk. Lymphoma. 35:283-288, 1999; Melki et al., Cancer Res. 59:3730-3740, 1999).

Second, an individual tumor within a single patient has a unique epigenetic fingerprint reflective of the evolution of that tumor as compared to a tumor of the same type in a different patient (Costello et al., Nat. Genet. 24:132-138, 2000).

Generally, however, most studies of epigenetic alterations in cancer have focused primarily on either a very small set of known genes (Jones & Laird, Nat Genet. 21:163-167, 1999; Baylin & Herman, Trends Genet. 16:168-174, 2000) or on the global analysis of unknown CpG islands (Costello et al., Nat. Genet. 24:132-138, 2000), and thus do not provide a suitable diagnostic and/or prognostic framework.

Esophageal adenocarcinoma (“EAC”). Esophageal adenocarcinoma (“EAC”) arises from a multistep process whereby normal squamous mucosa undergoes metaplasia to specialized columnar epithelium (Intestinal Metaplasia (IM) or Barrett's esophagus), which then ultimately progresses to dysplasia and subsequent malignancy (Barrett et al., Nat. Genet. 22:106-109, 1999; Zhuang et al., Cancer Res. 56:1961-4, 1996). The incidence of EAC has increased rapidly in the Western World over the past three decades (Devesa et al., Cancer. 83:2049-2053, 1998; Jankowski et al., Am. J. Pathol. 154:965-973, 1999).

Unfortunately, epigenetic studies of this model have so far been limited to the DNA methylation analysis of a few genes (Wong et al., Cancer Res. 57:2619-2622, 1997; Klump et al., Gastroenterology. 115:1381-1386, 1998; Eads et al., Cancer Res. 60:5021-5026,2000).

CpG island methylator phenotype (“CIMP”). It has previously been reported that a subset of colorectal and gastric tumors display a CpG island methylator phenotype (“CIMP”), characterized by widespread, aberrant hypermethylation changes affecting multiple loci in a single tumor (Toyota et al., Proc. Natl. Acad. Sci. USA 96:8681-8686, 1999; Toyota et al., Cancer Res. 59:5438-5442, 1999). This is reflected in a bimodal distribution of the frequency of the number of genes methylated in a group of tumors (Toyota et al., Proc. Natl. Acad. Sci. USA 96:8681-8686, 1999). CIMP tumors are a distinct group of tumors that are defined by a high degree of concordant CpG island hypermethylation of genes exclusively methylated in cancer, or type C genes. CIMP is now thought to be a new, distinct, yet major pathway of tumorigenesis (Toyota et al., Proc. Natl. Acad. Sci. USA 96:8681-8686, 1999; Toyota et al., Cancer Res. 59:5438-5442, 1999).

However, the role, if any, of the CIMP pathway in the tumor evolution of EAC is still uncharacterized, because the previous epigenetic studies only analyzed one (Wong et al., Cancer Res. 57:2619-2622, 1997; Klump et al., Gastroenterology. 115:1381-1386, 1998) or a few genes (Eads et al., Cancer Res. 60:5021-5026, 2000).

Therefore, there is a need in the art for novel methods of cancer detection, chemoprediction and prognostics. There is a need in the art to define novel coordinate patterns of CpG island methylation changes at multiple loci during different steps of a disease, such as cancer. There is a need in the art to determine tumor-type-specific, and patient-specific epigenetic patterns or fingerprints. There is a need in the art to provide biomarkers or probes, such as EAC-specific biomarkers or probes, that can be used in diagnostic and/or prognostic methods for the treatment of cancer. There is a need in the art to determine whether esophageal adenocarcinoma displays a CIMP. There is a need in the art for novel methods for determining the stage of a tumor. The present invention addresses these needs.

### SUMMARY OF THE INVENTION

The present invention provides a method for diagnosing cancer or cancer-related conditions from tissue samples, comprising: (a) obtaining a tissue sample from a test tissue or region to be diagnosed; (b) performing a methylation assay of the tissue sample, wherein the methylation assay determines the methylation state of genomic CpG sequences, wherein the genomic CpG sequences are located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR, and combinations thereof; and (c) making a diagnostic or prognostic prediction of the cancer based, at least in part, upon the methylation state of the genomic CpG sequences. Preferably, the genomic CpG sequences located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS, correspond to genomic CpG sequences of CpG islands. Preferably, the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR gene sequences are those defined by the specific oligonucleotide primers and probes corresponding to SEQ ID Nos: 1-60, 64 and 65, as listed in TABLE II, or portions thereof. Preferably, the CpG islands are located within the promoter regions of the genes. Preferably, the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, and TYMS gene sequences correspond to any CpG island sequences associated with the sequences defined by the specific oligonucleotide primers and probes corresponding to SEQ ID Nos:1-54, 58-60, 64 and 65, as listed in TABLE II, or portions thereof, wherein the associated CpG island sequences are those contiguous sequences of genomic DNA that encompass at least one nucleotide of the sequences defined by the specific oligonucleotide primers and probes corresponding to SEQ ID Nos:1-54, 58-60, 64 and 65, and satisfy the criteria of having both a frequency of CpG dinucleotides corresponding to an Observed/Expected Ratio>0.6, and a GC Content>0.5.

Preferably, the genomic CpG sequences are located within at least one gene sequence selected from the group consisting of APC, CDKN2A, MYOD1, CALCA, ESR1, MGMT and TIMP3, and combinations thereof. Preferably, the genomic CpG sequences located within at least one gene sequence selected from the group consisting of APC, CDKN2A, MYOD1, CALCA, ESR1, MGMT and TIMP3, correspond to genomic CpG sequences of CpG islands. Preferably, the APC, CDKN2A, MYOD1, CALCA, ESR1, MGMT and TIMP3 gene sequences are those defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, respectively, as listed in TABLE II. Preferably, the CpG islands are located within the promoter regions of the genes. Preferably, the APC, CDKN2A, MYODI, CALCA, ESRI, MGMT and TIMP3 gene sequences correspond to any CpG island sequences associated with the sequences defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, respectively, as listed in TABLE II, or portions thereof, wherein the associated CpG island sequences are those contiguous sequences of genomic DNA that encompass at least one nucleotide of the sequences defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, and satisfy the criteria of having both a frequency of CpG dinucleotides corresponding to an Observed/Expected Ratio>0.6, and a GC Content>0.5.

Preferably, the cancer or cancer-related condition is selected from the group consisting of gastrointestinal or esophageal adenocarcinoma, gastrointestinal or esophageal dysplasia, gastrointestinal or esophageal metaplasia, Barrett's intestinal tissue, pre-cancerous conditions in normal esophageal squamous mucosa, and combinations thereof. Preferably, the cancer is esophageal adenocarcinoma, and wherein making a diagnostic or prognostic prediction of the cancer, based upon the methylation state of the genomic CpG sequences provides for classification of the adenocarcinoma by grade or stage.

Preferably, the methylation assay used to determine the methylation state of genomic CpG sequences is selected from the group consisting of “MethylLight™”, MS-SNuPE, MSP, COBRA, MCA, and DMH, and combinations thereof.

Preferably, the methylation assay used to determine the methylation state of genomic CpG sequences is based, at least in part, on an array or microarray comprising CpG sequences located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR. Preferably, the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, and TYMS gene sequences correspond to any CpG isiand sequences associated with the sequences defined by the specific oligonucleotide primers and probes corresponding to SEQ ID Nos:1-54, 58-60, 64 and 65, as listed in TABLE II, or portions thereof, wherein the associated CpG island sequences are those contiguous sequences of genomic DNA that encompass at least one nucleotide of the sequences defined by the specific oligonucleotide primers and probes corresponding to SEQ ID Nos:1-54, 58-60, 64 and 65, and satisfy the criteria of having both a frequency of CpG dinucleotides corresponding to an Observed/Expected Ratio>0.6, and a GC Content>0.5. Preferably, the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR gene sequences are those defined by, or correspond to the specific oligonucleotide primers and probes corresponding to SEQ ID Nos:1-60, 64 and 65, as listed in TABLE II, or portions thereof.

Preferably, the methylation state of genomic CpG sequences that is determined is that of hypermethylation, hypomethylation or normal methylation.

The present invention also provides a kit useful for diagnosis or prognosis of cancer or cancer-related conditions, comprising a carrier means containing one or more containers comprising: (a) a container containing a probe or primer which hybridizes to any region of a sequence located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR; and (b) additional standard methylation assay reagents required to affect detection of methylated CpG-containing nucleic acid based, at least in part, on the probe or primer. Preferably, the additional standard methylation assay reagents are standard reagents for performing a methylation assay from the group consisting of MethyLight™, MS-SNuPE, MSP, COBRA, MCA and DMH, and combinations thereof. Preferably, the probe or primer comprises at least about 12 to 15 nucleotides of a sequence selected from the group consisting of SEQ ID Nos:1-60, 64 and 65, as listed in TABLE II.

The present invention further provides a kit useful for diagnosis or prognosis of cancer or cancer-related conditions, comprising a carrier means containing one or more containers comprising: (a) an array or micorarray comprising sequences of at least about 12 to 15 nucleotides of a sequence selected from the group consisting of SEQ ID Nos:1-60, 64, 65, and any sequence located within a CpG island sequence associated with SEQ ID NOs:1-54, 58-60, 64 and 65.

### BRIEF DESCRIPTION OF THE DRAWINGS

FIG. 1 shows, according to the present invention, a quantitative methylation analysis of a panel of 20 genes from a screen of 84 tissue specimens from 31 patients with different stages of Barrett's esophagus (“IM”), dysplasia (“DYS”) and/or associated esophageal adenocarcinoma (“T”). Methylation analysis was performed using the MethyLight™ assay (Eads et al., Cancer Res. 59:2302-2306, 1999; Eads et al., Nucleic Acids Res. 28:E32, 2000). The percentage of fully methylated molecules at a specific locus (PMR=Percent of Methylated Reference) was calculated by dividing the GENE/ACTB ratio of a sample by the GENE/ACTB ratio of SssI-treated sperm DNA and multipling by 100. The resulting percentages were then dichotomized at 4% PMR to facilitate graphical representation and to reveal tissue-specific patterns (as described herein). “N” indicates an analysis for which the control gene ACTB did not reach sufficient levels to allow the detection of a minimal value of 1 PMR for that methylation reaction in that particular sample.

FIG. 2 shows the percent of samples methylated for each gene by tissue type. The data was dichotomized at 4 PMR, with 4 PMR and higher designated as methylated, and below 4 PMR as unmethylated. The genes, according to the present invention, were grouped according to their respective epigenetic gene classes (A-G) as shown in FIG. 1. The letter “n” equals the number of samples analyzed for each tissue.

FIG. 3 shows a comparison of epigenetic profiles according to the present invention. The data was dichotomized at 4 PMR, with 4 PMR and higher designated as methylated, and below 4 PMR as unmethylated. Error bars represent the standard error of the mean. Top panel: Mean percent of genes methylated in each gene Class (A-F or ALL 19 CpG islands) by tissue type (N, normal esophagus; S, stomach; IM, intestinal metaplasia; DYS, dysplasia; T, adenocarcinoma). The error bars represent the standard error of the mean (SEM). Bottom panel: Statistical analysis of the difference in mean percent of genes methylated in different tissues by gene Class (A-F) or for all 19 CpG islands combined (ALL). The p-values were generated by a Fisher's Protected Least Significant Difference (PLSD) test, adapted for use with unequal sample numbers (SAS Statview™ software).

FIG. 4 shows the relationship between Class A methylation frequency and tumor stage according to the present invention. The data was dichotomized at 4 PMR, with 4 PMR and higher designated as methylated, and below 4 PMR as unmethylated. Upper panel: Mean number of genes methylated for Class A with respect to tumor stage (I-IV) is shown (see FIG. 1). The error bars represent the standard error of the mean (SEM). The letter “n” equals the number of samples analyzed in each tumor stage. Lower panel: Statistical analysis of the difference in mean number of Class A genes methylated by tumor stage. The p-values were generated by a Fisher's Protected Least Significant Difference (PLSD) test, adapted for use with unequal sample numbers (SAS Statview™ software).

FIG. 5 shows, according to the present invention, the percent of two or more Class A genes methylated in intestinal metaplasia (“IM”) tissues with (“Y”), or without (“N”) associated dysplasia and/or adenocarcinoma. The data was dichotomized at 4 PMR, with 4 PMR and higher designated as methylated, and below 4 PMR as unmethylated. Left panel: Class A methylation in the IM data illustrated in FIG. 1. Right panel: Class A methylation in the IM for a completely independent follow-up study of twenty different microdissected IM samples. The error bars represent the standard error of the mean (SEM). The letter “n” equals the number of samples analyzed in each tissue group.

FIG. 6 shows, according to the present invention, methylation frequency distributions in the progression of esophageal adenocarcinoma. The data was dichotomized at 4 PMR, with 4 PMR and higher designated as methylated, and below 4 PMR as unmethylated. The proportion of patients with zero to three (Class A), zero to nine (Classes A+D) and zero to fourteen CpG islands (Classes A+B+C+D) methylated in each tissue is shown. Class E and F CpG islands were not included since there was no variation in the frequency of methylation between the different tissue. The letter “n” equals the number of samples analyzed in each tissue.

### DETAILED DESCRIPTION OF THE INVENTION

Definitions:

The term “EAC” refers to esophageal adenocarcinoma, but also encompasses different histological stages of esophageal adenocarcinoma corresponding to a multistep process whereby normal squamous mucosa undergoes metaplasia to specialized columnar epithelium (Intestinal Metaplasia (IM) or Barrett's esophagus), which then ultimately progresses to dysplasia and subsequent malignancy (Barrett et al., Nat. Genet. 22:106-109, 1999; Zhuang et al., Cancer Res. 56:19614, 1996);

The term “CIMP” refers to CpG island methylator phenotype, characterized by widespread aberrant hypermethylation changes affecting multiple loci in a single tumor. This is reflected in a bimodal distribution of the frequency of the number of genes methylated in a group of tumors (16). CIMP tumors are a distinct group of tumors that are defined by a high degree of concordant CpG island hypermethylation of genes exclusively methylated in cancer, or type C genes. CIMP is now thought to be a new, distinct, yet major pathway of tumorigenesis (Toyota et al., Proc. Natl. Acad. Sci. USA 96:8681-8686, 1999; Toyota et al., Cancer Res. 59:5438-5442, 1999) (see “Background,” above);

The term “PMR” refers to percent of methylated reference, and is calculated as described herein under Example I;

“GC Content” refers, within a particular DNA sequence, to the [(number of C bases+number of G bases)/band length for each fragment];

“Observed/Expected Ratio” (“O/E Ratio”) refers to the frequency of CpG dinucleotides within a particular DNA sequence, and corresponds to the [number of CpG sites/(number of C bases×number of G bases)]×band length for each fragment;

“CpG Island” refers to a contiguous region of genomic DNA that satisfies the criteria of (1) having a frequency of CpG dinucleotides corresponding to an “Observed/Expected Ratio”>0.6), and (2) having a “GC Content”>0.5. CpG islands are typically, but not always, between about 0.2 to about 1 kb in length. A CpG island sequence associated with a particular SEQ ID NO sequence of the present invention is that contiguous sequence of genomic DNA that encompasses at least one nucleotide of the particular SEQ ID NO sequence, and satisfies the criteria of having both a frequency of CpG dinucleotides corresponding to an Observed/Expected Ratio>0.6), and a GC Content>0.5;

“Methylation state” refers to the presence or absence of 5-methylcytosine (“5-mCyt”) at one or a plurality of CpG dinucleotides within a DNA sequence;

“Hypermethylation” refers to the methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample;

“Hypomethylation” refers to the methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample;

“Methylation assay” refers to any assay for determining the methylation state of a CpG dinucleotide within a sequence of DNA;

“MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997;

“MethyLight” refers to the art-recognized fluorescence-based real-time PCR technique described by Eads et al., Cancer Res. 59:2302-2306, 1999;

“Ms-SNuPE” (Methylation-sensitive Single Nucleotide Primer Extension) refers to the art-recognized assay described by Gonzalgo & Jones, Nucleic Acids Res. 25:2529-2531, 1997;

“MSP” (Methylation-specific PCR) refers to the art-recognized methylation assay described by Herman et al. Proc. Natl. Acad. Sci. USA 93:9821-9826, 1996, and by U.S. Pat. No. 5,786,146;

“COBRA” (Combined Bisulfite Restriction Analysis) refers to the art-recognized methylation assay described by Xiong & Laird, Nucleic Acids Res. 25:2532-2534, 1997;

“MCA” (Methylated CpG Island Amplification) refers to the methylation assay described by Toyota et al., Cancer Res. 59:2307-12, 1999, and in WO 00/26401A1;

“DMH” (Differential Methylation Hybridization) refers to the art-recognized methylation assay described in Huang et al., Hum. Mol. Genet., 8:459-470, 1999, and in Yan et al., Clin. Cancer Res. 6:1432-38, 2000;

Genes and Associated Literature References:

“APC” refers to the adenomatous polyposis coli gene (Eads et al., Cancer Res. 59:2302-2306, 1999; Hiltunen et al., Int. J. Cancer. 70:644-648, 1997);

“ARF” refers to the P14 cell cycle regulator, tumor suppressor gene (Esteller et al., Cancer Res. 60:129-133, 2000; Robertson & Jones, Mol. Cell. Biol. 18:6457-6473, 1998);

“CALCA” refers to the calcitonin gene (Melki et al., Cancer Res. 59:3730-3740, 1999; Hakkarainen et al., Int. J. Cancer. 69:471-474, 1996);

“CDHI” refers to the E-cadherin gene (Melki et al., Cancer Res. 59:3730-3740, 1999; Ueki et al., Cancer Res. 60:1835-1839, 2000);

“CDKN2A” refers to the P16 gene (Jones & Laird, Nat. Genet. 21:163-167, 1999; Melki et al., Cancer Res. 59:3730-3740, 1999; Baylin & Herman, Trends Genet. 16:168-174, 2000; Cameron et al., Nat. Genet. 21:103-107, 1999; Ueki et al., Cancer Res. 60:1835-1839, 2000);

“CDKN2B” refers to the P15 gene (Meki et al., Cancer Res. 59:3730-3740, 1999; Cameron et al., Nat. Genet. 21:103-107, 1999);

“CTNNB1” refers to the beta-catenin gene;

“ESR1” refers to the estrogen receptor alpha gene (Jones & Laird, Nat. Genet. 21:163-167, 1999; Baylin & Herman, Trends Genet. 16:168-174, 2000);

“GSTP1,” refers to the glutathione S-transferase P1 gene (Melki et al., Cancer Res. 59:3730-3740, 1999; Tchou et al., Int. J. Oncol. 16:663-676, 2000);

“HIC1” refers to the hypermethylated in cancer 1 gene (Melki et al., Cancer Res. 59:3730-3740, 1999; Wales et al., Nat. Med. 1:570-577, 1995);

“MGMT” refers to the O6-methylguanine-DNA methyltransferase gene (Esteller et al., Cancer Res. 59:793-797, 1999);

“MLH1” refers to the Mut L homologue 1 gene (Jones & Laird, Nat. Genet. 21:163-167, 1999; Baylin & Herman, Trends Genet. 16:168-174, 2000; Cameron et al., Nat. Genet. 21:103-107, 1999; Esteller et al., Am. J. Pathol. 155:1767-1772, 1999, Ueki et al., Cancer Res. 60:1835-1839, 2000);

“MTHFR” refers to the methyl-tetrahydrofolate reductase gene (Pereira et al., Oncol. Rep. 6:597-599, 1999);

“MYOD1” refers to the myogenic determinant 1 gene (Eads et al., Cancer Res. 59:2302-2306, 1999; Cheng et al., Br. J Cancer. 75:396-402, 1997);

“PTGS2” refers to the cyclooxygenase 2 gene (Zimmermann et al., Cancer Res. 59:198-204, 1999);

“RB1” refers to the retinoblastoma gene (Stirzaker et al., Cancer Res. 57:2229-2237, 1997; Sakai et al., Am. J. Hum. Genet. 48:880-888, 1991);

“TGFBR2” refers to the transforming growth factor beta receptor II gene (Kang et al., Oncogene. 18:7280-7286, 1999; Hougaard et al., Br. J. Cancer. 79:1005-1011, 1999);

“THBS1” refers to the thrombospondin 1 gene (Ueki et al., Cancer Res. 60:1835-1839, 2000; Li et al., Oncogene. 18:284-3289, 1999);

“TIMP3” refers to the tissue inhibitor of metallinoproteinase 3 gene (Cameron et al., Nat. Genet. 21:103-107, 1999; Ueki et al., Cancer Res. 60:1835-1839, 2000; Bachman et al., Cancer Res. 59:798-802, 1999);

“TYMS1” refers to the thymidylate synthetase gene (Sakamoto et al., In: L. Herrera (ed.) Familial adenomatous polyposis, pp. 315-324. New York: Alan R. Liss, 1990).

Overview

The present invention encompasses a broad, multi-gene approach that provides novel and therapeutically useful insight into concordant methylation behavior between and among genes. In particular embodiments, the present invention provides novel epigenomic fingerprints for the different histological stages of esophageal adenocarcinoma (EAC).

More specifically, the present invention combines the advantages of both targeted and comprehensive approaches by analyzing 20 different genes (see Table 1, below) using a quantitative, high-throughput methylation assay, “MethyLight™” (Eads et al., Cancer Res. 59:2302-2306, 1999; Eads et al., Cancer Res. 60:5021-5026, 2000; Eads et al., Nucleic Acids Res. 28:E32, 2000), to (i) more extensively characterize the methylation changes in esophageal adenocarcinoma (EAC); to (ii) generate epigenomic fingerprints for the different histological stages of EAC; to (iii) identify epigenetic biomarkers useful in disease diagnosis and prevention; and to (iv) determine if CIMP is a contributor to the tumorigenesis of esophageal adenocarcinoma tumors.

A total of 104 tissue specimens from 51 patients with different stages of Barrett's esophagus and/or associated adenocarcinoma were analyzed. Specifically, 84 of these tissue specimens were screened with the full panel of 20 genes, revealing distinct classes of methylation patterns in the different types of tissue.

The most informative genes, for purposes of the present invention, were those with an intermediate frequency of significant hypermethylation (i.e., those ranging from about 15% (CDKN2A) to about 60% (MGMT) of the samples). This group of genes could be further subdivided into three classes, according to the (1) absence (CDKN2A, ESR1 and MYOD1), or (2) presence (CALCA, MGMT and TIMP3) of methylation in normal esophageal mucosa and stomach, or (3) the infrequent methylation of normal esophageal mucosa accompanied by methylation in all normal stomach samples (APC).

The other genes were relatively less informative, since the frequency of hypermethylation was below about 5% (ARF, CDH1, CDKN2B, GSTP1, MLH1, PTGS2 and THBS1), completely absent (CTNNB1, RB1, TGFBR2 and TYMS1) or ubiquitous (HIC1 and MTHFR), regardless of tissue type.

Each class of gene undergoes unique epigenetic changes at different steps of disease progression of EAC, consistent with a step-wise loss of multiple protective barriers against CpG island hypermethylation. The aberrant hypermethylation occurs at many different loci in the same tissues, consistent with an overall deregulation of methylation control in EAC tumorigenesis. However, there was no clear evidence for a distinct group of tumors with a CpG island methylator phenotype (“CIMP”).

Additionally, normal and metaplastic tissues from patients with evidence of associated dysplasia or cancer displayed a significantly higher incidence of hypermethylation than similar tissues from patients with no further progression of their disease. The fact that the samples from these two groups of patients were histologically indistinguishable, yet molecularly distinct, indicates, according to the present invention, that the occurrence of such hypermethylation provides a novel and valuable clinical tool to identify patients with pre-malignant Barrett's, who are at risk for further progression.

TABLE I shows a list of gene names and functions analyzed by the MethyLight™ assay in EAC. The genes are listed in alphabetical order based on their designated HUGO (HUman Genome Organization) names. The genes are divided into three groups according to whether or not they have CpG islands and are known to be methylated in other tumors. A brief description of the function of each gene is included.

TABLE I
List of Gene Names Analyzed by the MethyLight ™ assay in EAC

| | | | |
|---|---|---|---|
| GENE | | TUMORS WITH | PROPOSED |
| SYMBOL | NAME | HYPERMETHYLATION^{†} | FUNCTION |

CPG ISLAND: METHYLATED IN TUMORS

| | | | |
|---|---|---|---|
| APC | Adenomatous Polyposis Coli | Colon | WNT Signaling Pathway; Beta- |
| | | | Catenin Degradation; Tumor |
| | | | Suppressor |
| ARF | P14 | Colon, Lymphoma | Cell Cycle Regulator; Tumor |
| | | | Suppressor |
| CALCA | Calcitonin | Breast, Colon, Leukemia | Regulates Calcium Levels via |
| | | | Adenylate Cyclase |
| CDH1 | E-Cadherin | AML, Bladder, Breast, Colon, Gastric, | Cell Adhesion |
| | | Thyroid | |
| CDKN2A | P16 | AML, Bladder, Colon, Gastric, | Cell Cycle Regulator, Tumor |
| | | Lymphoma, Melanoma | Suppressor |
| CDKN2B | P15 | Colon, Hematological Malignancies | Cell Cycle Regulator, Tumor |
| | | | Suppressor |
| ESR1 | Estrogen Receptor Alpha | AML, Colon, Breast, Lung, Leukemia | Hormone Receptor in Mammary |
| | | | Cells; Growth Suppressive in |
| | | | Colorectal Cancer Cells |
| GSTP1 | Glutathione S-Transferase PI | Breast, Prostate, Hepatocellular | Protects Against Oxidant and |
| | | | Electrophilic Carcinogens |
| HIC1 | Hypermethylated in Cancer 1 | Brain, Breast, Colon, Renal, Leukemia, | Zinc Finger Transcription Factor; |
| | | Lymphoma | Potential Tumor Suppressor |
| MGMT | O6-Methylguanine-DNA | Brain, Colon, Lymphoma, Non-Small Cell | DNA Repair |
| | Methyltransferase | Lung Cancer | |
| MLH1 | Mut L Homologue 1 | Colon, Endometrial, Gastric | DNA Mismatch Repair |
| MYOD1 | Myogenic Determinant 1 | Breast, Colon, Ovarian | Muscle Differentiation |
| RB1 | Retinoblastoma 1 | Retinoblastoma | Cell Cycle Regulator; Tumor |
| | | | Suppressor |
| TGFBR2 | Transforming Growth Factor | Colon, Gastric, Small-Cell Lung Cancer | Serine-Threonine Kinase |
| | Beta Receptor II | | Receptor; Cell Signaling |
| THBS1 | Thrombospondin 1 | Colon, Glioblastomas | Angiogenesis Inhibitor |
| TIMP3 | Tissue Inhibitor of | Brain, Breast, Colon, Kidney, Lung, | Metastasis, Angiogenesis |
| | Metallinoproteinase 3 | Pancreatic | |

CPG ISLAND: UNKNOWN METHYLATION STATUS

| | | | |
|---|---|---|---|
| CTNNB1 | Beta-Catenin | Unknown | WNT Signaling Pathway |
| | | | (Regulated by APC); Cell |
| | | | Adhesion via E-Cadherin |
| PTGS2 | Cyclooxygenase 2 | Unknown | Prostaglandin Synthesis from |
| | | | Arachidonic Acid |
| TYMS | Thymidylate Synthetase | Unknown | Nucleotide/DNA Synthesis |

NON-CPG ISLAND: UNKNOWN METHYLATION STATUS

| | | | |
|---|---|---|---|
| MTHFR | Methyl-Tetetrahydrofolate | Unknown | Folate Metabolism, Methionine |
| | Reductase | | Synthesis; Predisposing |
| | | | Polymorphism in Colon Cancer |

†See literature references relating to specific genes under “DEFINITIONS,” herein above.

Diagnostic and Prognostic Assays for Cancer

The present invention provides for diagnostic and prognostic cancer assays based on determination of the methylation state of one or more of the disclosed 20 gene sequences (APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR; see TABLES I and II, below; and see under “Definitions,” above), or methylation-altered DNA sequence embodiments thereof. These 20 gene sequence regions are defined herein by the oligomeric primers and probes corresponding to SEQ ID NOS:1-60, 64 and 65 (see TABLE II, below). SEQ ID NOS:61-63 correspond to the ACTB “control” gene region used in the present analysis (see EXAMPLE 1, below).

Additionally, 19 of these 20 gene sequence regions correspond to CpG islands or regions thereof (based on GC Content and O/E ratio); namely APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS (see TABLE 1, below). Thus, based on the fact that the methylation state of a portion of a given CpG island is generally representative of the island as a whole, the present invention further encompasses the novel use of any sequences within the 19 complete CpG islands associated with these 19 gene sequence regions (defined herein by the primers and probes corresponding to SEQ ID NOS:1-60, 64 and 65 (see TABLE II, below) in cancer prognostic and diagnostic applications), where a CpG island sequence associated with one of these 19 gene sequences is that contiguous sequence of genomic DNA that encompasses at least one nucleotide of one of these 19 gene sequences, and satisfies the criteria of having both a frequency of CpG dinucleotides corresponding to an Observed/Expected Ratio>0.6, and a GC Content>0.5.

Typically, such assays involve obtaining a tissue sample from a test tissue, performing a methylation assay on DNA derived from the tissue sample to determine the associated methylation state, and making a diagnosis or prognosis based thereon.

The methylation assay is used to determine the methylation state of one or a plurality of CpG dinucleotide within a DNA sequence of the DNA sample. According to the present invention, possible methylation states include hypermethylation and hypomethylation, relative to a normal state (i.e., non-cancerous control state). Hypermethylation and hypomethylation refer to the methylation states corresponding to an increased or decreased, respectively, presence of 5-methylcytosine (“5-mCyt”) at one or a plurality of CpG dinucleotides within a DNA sequence of the test sample, relative to the amount of 5-mCyt found at corresponding CpG dinucleotides within a normal control DNA sample.

A diagnosis or prognosis is based, at least in part, upon the determined methylation state of the sample DNA sequence compared to control data obtained from normal, non-cancerous tissue.

Methylation Assay Procedures

Various methylation assay procedures are known in the art, and can be used in conjunction with the present invention. These assays allow for determination of the methylation state of one or a plurality of CpG dinucleotides within a DNA sequence (e.g., CpG islands). Such assays involve, among other techniques, DNA sequencing of bisulfite-treated DNA, PCR (for sequence-specific amplification), Southern blot analysis, use of methylation-sensitive restriction enzymes, etc.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

Preferably, assays such as “MethyLight™” (a fluorescence-based real-time PCR technique) (Eads et al., Cancer Res. 59:2302-2306, 1999), Methylation-sensitive Single Nucleotide Primer Extension reactions (“Ms-SnuPE”; Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”;Toyota et al., Cancer Res. 59:2307-12, 1999) are used alone or in combination with other of these methods. Methylation assays that can be used in various embodiments of the present invention include, but are not limited to, the following assays.

COBRA (Combined Bisulfite Restriction Analysis). COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. Additioinally, this technique can be reliably applied to DNA obtained from microdissected paraffin-embedded tissue samples. Typical reagents (e.g., as might be found in a typical COBRA-based methylation kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides (although other label schemes known in the art including, but not limited, to fluorescent and phosphorescent schemes can be used). Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Ms-SnuPE (Methylation-sensitive Single Nucleotide Primer Extension). The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites. Typical reagents (e.g., as might be found in a typical Ms-SNuPE-based methylation kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP (Methylation-specific PCR). MSP allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical -MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

MCA (Methylated CpG Island Amplification). The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

DMH (Differential Methylation Hybridization). DMH refers to the art-recognized, array-based methylation assay described in Huang et al., Hum. Mol. Genet., 8:459-470, 1999, and in Yan et al., Clin. Cancer Res. 6:1432-38, 2000. DMH allows for a genome-wide screening of CpG island hypermethylation in cancer cell lines, and. Briefly, CpG island tags are arrayed on solid supports (e.g., nylon membranes, silicon, etc.), and probed with “amplicons” representing a pool of methylated CpG DNA, from test (e.g., tumor) or reference samples. The differences in test and reference signal intensities on screened CpG island arrays reflect methylation alterations of corresponding sequences in the test DNA.

MethyLight™. In preferred embodiments, the MethyLight™ assay is used to determine the methylation status of one or more CpG sequences. The MethyLight™ assay is a high-throughput quantitative methylation assay that utilizes fluorescence-based real-time PCR (TaqMan®) technology that requires no further manipulations after the PCR step (Eads et al., Cancer Res. 60:5021-5026, 2000; Eads et al., Cancer Res. 59:2302-2306, 1999; Eads et al., Nucleic Acids Res. 28:E32, 2000). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may assay be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based methylation kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase. A detailed description of four alternate process applications (“A” through “D”) of the MethyLight™ assay follows below. Preferably, the quantitative MethyLight™ process application “B” is used.

MethyLight™-based detection of the methylated nucleic acid is relatively rapid and is based on amplification-mediated displacement of specific oligonucleotide probes. In a preferred embodiment, amplification and detection, in fact, occur simultaneously as measured by fluorescence-based real-time quantitative PCR (“RT-PCR”) using specific, dual-labeled TaqMan® oligonucleotide probes, with no requirement for subsequent manipulation or analysis. The displaceable probes can be specifically designed to distinguish between methylated and unmethylated CpG sites present in the original, unmodified nucleic acid sample.

Like the technique of methylation-specific PCR (“MSP”; U.S. Pat. No. 5,786,146), MethyLight™ provides for significant advantages over previous PCR-based and other methods (e.g., Southern analyses) used for determining methylation patterns. MethyLight™ is substantially more sensitive than Southern analysis, and facilitates the detection of a low number (percentage) of methylated alleles in very small nucleic acid samples, as well as paraffin-embedded samples. Moreover, in the case of genomic DNA, analysis is not limited to DNA sequences recognized by methylation-sensitive restriction endonucleases, thus allowing for fine mapping of methylation patterns across broader CpG-rich regions. MethyLight™ also eliminates any false-positive results, that otherwise might result from incomplete digestion by methylation-sensitive restriction enzymes, inherent in previous PCR-based methylation methods.

MethyLight™ can be applied as a quantitative process for measuring methylation amounts, and is substantially more rapid than other methods. MethyLigh™ does not require any post-PCR manipulation or processing. This not only greatly reduces the amount of labor involved in the analysis of bisulfite-treated DNA, but it also provides a means to avoid handling of PCR products that could contaminate future reactions.

One process embodiment uses MethyLight™ for the unbiased amplification of all possible methylation states using primers that do not cover any CpG sequences in the original, unmodified DNA sequence. To the extent that all methylation patterns are amplified equally, quantitative information about DNA methylation patterns are then distilled from the resulting PCR pool by any technique capable of detecting sequence differences (e.g., by fluorescence-based PCR).

MethyLight™ employs one or a series of CpG-specific TaqMan® probes, each corresponding to a particular methylation site in a given amplified DNA region, are constructed. This series of probes is then utilized in parallel amplification reactions, using aliquots of a single, modified DNA sample, to simultaneously determine the complete methylation pattern present in the original unmodified sample of genomic DNA. This is accomplished in a fraction of the time and expense required for direct sequencing of the sample of genomic DNA, and are substantially more sensitive. Moreover, one embodiment of MethyLight™ provides for a quantitative assessment of such a methylation pattern.

The present invention, as described herein, may be practiced using a variety of methylation assays. For MethyLight™ emabodiments, there are four process techniques and associated diagnostic kits that a methylation-dependent nucleic acid modifying agent (e.g., bisulfite), to both qualitatively and quantitatively determine CpG methylation status in nucleic acid samples (e.g., genomic DNA samples). The four processes are described herein as processes “A,” “B,” “C” and “D.” Overall, methylated-CpG sequence discrimination is designed to occur at the level of amplification, probe hybridization or at both levels. For example, applications C and D utilize “biased” primers that distinguish between modified unmethylated and methylated nucleic acid and provide methylated-CpG sequence discrimination at the PCR amplification level. Process B uses “unbiased” primers (that do not cover CpG methylation sites), to provide for unbiased amplification of modified nucleic acid, but rather utilize probes that distinguish between modified unmethylated and methylated nucleic acid to provide for quantitative methylated-CpG sequence discrimination at the detection level (e.g., at the fluorescent (or luminescent) probe hybridization level only). Process A does not, in itself, provide for methylated-CpG sequence discrimination at either the amplification or detection levels, but supports and validates the other three applications by providing control reactions for input DNA.

MethyLight™ Process D. In a first MethyLight™ embodiment, the invention provides a method for qualitatively detecting a methylated CpG-containing nucleic acid, the method including: contacting a nucleic acid-containing sample with a modifying agent that modifies unmethylated cytosine to produce a converted nucleic acid; amplifying the converted nucleic acid by means of two oligonucleotide primers in the presence of a specific oligonucleotide hybridization probe, wherein both the primers and probe distinguish between modified unmethylated and methylated nucleic acid; and detecting the “methylated” nucleic acid based on amplification-mediated probe displacement.

The term “modifies” as used herein means the conversion of an unmethylated cytosine to another nucleotide by the modifying agent, said conversion distinguishing unmethylated from methylated cytosine in the original nucleic acid sample. Preferably, the agent modifies unmethylated cytosine to uracil. Preferably, the agent used for modifying unmethylated cytosine is sodium bisulfite, however, other equivalent modifying agents that selectively modify unmethylated cytosine, but not methylated cytosine, can be substituted in the method of the invention. Sodium-bisulfite readily reacts with the 5, 6-double bond of cytosine, but not with methylated cytosine, to produce a sulfonated cytosine intermediate that undergoes deamination under alkaline conditions to produce uracil. Because Taq polymerase recognizes uracil as thymine and 5-methylcytidine (^{m5}C) as cytidine, the sequential combination of sodium bisulfite treatment and PCR amplification results in the ultimate conversion of unmethylated cytosine residues to thymine (C→U→T) and methylated cytosine residues (“^{m}C”) to cytosine (^{m}C→^{m}C→C). Thus, sodium-bisulfite treatment of genomic DNA creates methylation-dependent sequence differences by converting unmethylated cyotsines to uracil, and upon PCR the resultant product contains cytosine only at positions where methylated cytosine-occurs in the unmodified nucleic acid.

Oligonucleotide “primers,” as used herein, means linear, single-stranded, oligomeric deoxyribonucleic or ribonucleic acid molecules capable of sequence-specific hybridization (annealing) with complementary strands of modified or unmodified nucleic acid. As used herein, the specific primers are preferably DNA. The primers of the invention embrace oligonucleotides of appropriate sequence and sufficient length so as to provide for specific and efficient initiation of polymerization (primer extension) during the amplification process. As used in the inventive processes, oligonucleotide primers typically contain 12-30 nucleotides or more, although may contain fewer nucleotides. Preferably, the primers contain from 18-30 nucleotides. The exact length will depend on multiple factors including temperature (during amplification), buffer, and nucleotide composition. Preferably, primers are single-stranded although double-stranded primers may be used if the strands are first separated. Primers may be prepared using any suitable method, such as conventional phosphotriester and phosphodiester methods or automated embodiments which are commonly known in the art.

As used in the inventive embodiments herein, the specific primers are preferably designed to be substantially complementary to each strand of the genomic locus of interest. Typically, one primer is complementary to the negative (−) strand of the locus (the “lower” strand of a horizontally situated double-stranded DNA molecule) and the other is complementary to the positve (+) strand (“upper” strand). As used in the embodiment of Application D, the primers are preferably designed to overlap potential sites of DNA methylation (CpG nucleotides) and specifically distinguish modified unmethylated from methylated DNA. Preferably, this sequence discrimination is based upon the differential annealing temperatures of perfectly matched, versus mismatched oligonucleotides. In the embodiment of Application D, primers are typically designed to overlap from one to several CpG sequences. Preferably, they are designed to overlap from 1 to 5 CpG sequences, and most preferably from 1 to 4 CpG sequences. By contrast, in a quantitative embodiment of the invention employed in the Examples of the present invention, the primers do not overlap any CpG sequences.

In the case of fully “unmethylated” (complementary to modified unmethylated nucleic acid strands) primer sets, the anti-sense primers contain adenosine residues (“A”s) in place of guanosine residues (“G”s) in the corresponding (−) strand sequence. These substituted As in the anti-sense primer will be complementary to the uracil and thymidine residues (“Us” and “Ts”) in the corresponding (+) strand region resulting from bisulfite modification of unmethylated C residues (“Cs”) and subsequent amplification. The sense primers, in this case, are preferably designed to be complementary to anti-sense primer extension products, and contain Ts in place of unmethylated Cs-in the corresponding (+) strand sequence. These substituted Ts in the sense primer will be complementary to the As, incorporated in the anti-sense primer extension products at positions complementary to modified Cs (Us) in the original (+) strand.

In the case of fully-methylated primers (complementary to methylated CpG-containing nucleic acid strands), the anti-sense primers will not contain As in place of Gs in the corresponding (−) strand sequence that are complementary to methylated Cs (i.e., mCpG sequences) in the original (+) strand. Similarly, the sense primers in this case will not contain Ts in place of methylated Cs in the corresponding (+) strand mCpG sequences. However, Cs that are not in CpG sequences in regions covered by the fully-methylated primers, and are not methylated, will be represented in the fully-methylated primer set as described above for unmethylated primers.

Preferably, as employed in the embodiment of process D, the amplification process provides for amplifying bisulfite converted nucleic acid by means of two oligonucleotide primers in the presence of a specific oligonucleotide hybridization probe. Both the primers and probe distinguish between modified unmethylated and methylated nucleic acid. Moreover, detecting the “methylated” nucleic acid is based upon amplification-mediated probe fluorescence. In one embodiment, the fluorescence is generated by probe degradation by 5′ to 3′ exonuclease activity of the polymerase enzyme. In another embodiment, the fluorescence is generated by fluorescence energy transfer effects between two adjacent hybridizing probes (Lightcycler® technology) or between a hybridizing probe and a primer. In another embodiment, the fluorescence is generated by the primer itself (Sunrise® technology). Preferably, the amplification process is an enzymatic chain reaction that uses the oligonucleotide primers to produce exponential quantities of amplification product, from a target locus, relative to the number of reaction steps involved.

As describe above, one member of a primer set is complementary to the (−) strand, while the other is complementary to the (+) strand. The primers are chosen to bracket the area of interest to be amplified; that is, the “amplicon.” Hybridization of the primers to denatured target nucleic acid followed by primer extension with a DNA polymerase and nucleotides, results in synthesis of new nucleic acid strands corresponding to the amplicon. Preferably, the DNA polymerase is Taq polymerase, as commonly used in the art. Although equivalent polymerases with a 5′ to 3′ nuclease activity can be substituted. Because the new amplicon sequences are also templates for the primers and polymerase, repeated cycles of denaturing, primer annealing, and extension results in exponential production of the amplicon. The product of the chain reaction is a discrete nucleic acid duplex, corresponding to the amplicon sequence, with termini defined by the ends of the specific primers employed. Preferably the amplification method used is that of PCR (Mullis et al., Cold Spring Harb. Symp. Quant. Biol. 51:263-273; Gibbs, Anal. Chem. 62:1202-1214, 1990), or more preferably, automated embodiments thereof which are commonly known in the art.

Preferably, methylation-dependent sequence differences are detected by methods based on fluorescence-based quantitative PCR (real-time quantitative PCR, Heid et al., Genome Res. 6:986-994, 1996; Gibson et al., Genome Res. 6:995-1001, 1996) (e.g., “TaqMan®,” “Lightcycler®,” and “Sunrise®” technologies). For the TaqMan® and Lightcycler® technologies, the sequence discrimination can occur at either or both of two steps: (1) the amplification step, or (2) the fluorescence detection step. In the case of the “Sunrise®” technology, the amplification and fluorescent steps are the same. In the case of the FRET hybridization, probes format on the Lightcycler®, either or both of the FRET oligonucleotides can be used to distinguish the sequence difference. Most preferably the amplification process, as employed in all inventive embodiments herein, is that of fluorescence-based Real Time Quantitative PCR (Heid et al., Genome Res. 6:986-994, 1996) employing a dual-labeled fluorescent oligonucleotide probe (TaqMan® PCR, using an ABI Prism 7700 Sequence Detection System, Perkin Elmer Applied Biosystems, Foster City, Calif.).

The “TaqMan®” PCR reaction uses a pair of amplification primers along with a nonextendible interrogating oligonucleotide, called a TaqMan® probe, that is designed to hybridize to a GC-rich sequence located between the forward and reverse (i.e., sense and anti-sense) primers. The TaqMan® probe further comprises a fluorescent “reporter moiety” and a “quencher moiety” covalently bound to linker moieties (e.g., phosphoramidites) attached to nucleotides of the TaqMan® oligonucleotide. Examples of suitable reporter and quencher molecules are: the 5′ fluorescent reporter dyes 6FAM (“FAM”; 2,7 dimethoxy-4,5-dichloro-6-carboxy-fluorescein), and TET (6-carboxy-4,7,2′,7′-tetrachlorofluorescein); and the 3′ quencher dye TAMRA (6-carboxytetramethylrhodamine) (Livak et al., PCR Methods Appl. 4:357-362, 1995; Gibson et al., Genome Res. 6:995-1001; and 1996; Heid et al., Genome Res. 6:986-994, 1996).

One process for designing appropriate TaqMan® probes involves utilizing a software facilitating tool, such as “Primer Express” that can determine the variables of CpG island location within GC-rich sequences to provide for at least a 110° C. melting temperature difference (relative to the primer melting temperatures) due to either specific sequence (tighter bonding of GC, relative to AT base pairs), or to primer length.

The TaqMan® probe may or may not cover known CpG methylation sites, depending on the particular inventive process used. Preferably, in the embodiment of process D, the TaqMan® probe is designed to distinguish between modified unmethylated and methylated nucleic acid by overlapping from 1 to 5 CpG sequences. As described above for the fully unmethylated and fully methylated primer sets, TaqMan® probes may be designed to be complementary to either unmodified nucleic acid, or, by appropriate base substitutions, to bisulfite-modified sequences that were either fully unmethylated or fully methylated in the original, unmodified nucleic acid sample.

Each oligonucleotide primer or probe in the TaqMan® PCR reaction can span anywhere from zero to many different CpG dinucleotides that each can result in two different sequence variations following bisulfite treatment (^{m}CpG, or UpG). For instance, if an oligonucleotide spans 3 CpG dinucleotides, then the number of possible sequence variants arising in the genomic DNA is 2³=8 different sequences. If the forward and reverse primer each span 3 CpGs and the probe oligonucleotide (or both oligonucleotides together in the case of the FRET format) spans another 3, then the total number of sequence permutations becomes 8×8×8=512. In theory, one could design separate PCR reactions to quantitatively analyze the relative amounts of each of these 512 sequence variants. In practice, a substantial amount of qualitative methylation information can be derived from the analysis of a much smaller number of sequence variants. Thus, in its most simple form, the inventive process can be performed by designing reactions for the fully methylated and the fully unmethylated variants that represent the most extreme sequence variants in a hypothetical example. The ratio between these two reactions, or alternatively the ratio between the methylated reaction and a control reaction (process A), would provide a measure for the level of DNA methylation at this locus.

Detection of methylation in the MethyLight™ embodiment of process D, as in other MethyLight™ embodiments herein, is based on amplification-mediated displacement of the probe. In theory, the process of probe displacement might be designed to leave the probe intact, or to result in probe digestion. Preferably, as used herein, displacement of the probe occurs by digestion of the probe during amplification. During the extension phase of the PCR cycle, the fluorescent hybridization probe is cleaved by the 5′ to 3′ nucleolytic activity of the DNA polymerase. On cleavage of the probe, the reporter moiety emission is no longer transferred efficiently to the quenching moiety, resulting in an increase of the reporter moiety fluorescent-emission spectrum at 518 nm. The fluorescent intensity of the quenching moiety (e.g., TAMRA), changes very little over the course of the PCR amplification. Several factors my influence the efficiency of TaqMan® PCR reactions including: magnesium and salt concentrations; reaction conditions (time and temperature); primer sequences; and PCR target size (i.e., amplicon size) and composition. Optimization of these factors to produce the optimum fluorescence intensity for a given genomic locus is obvious to one skilled in the art of PCR, and preferred conditions are further illustrated in the “Examples” herein. The amplicon may range in size from 50 to 8,000 base pairs, or larger, but may be smaller. Typically, the amplicon is from 100 to 1000 base pairs, and preferably is from 100 to 500 base pairs. Preferably, the reactions are monitored in real time by performing PCR amplification using 96-well optical trays and caps, and using a sequence detector (ABI Prism) to allow measurement of the fluorescent spectra of all 96 wells of the thermal cycler continuously during the PCR amplification. Preferably, process D is run in combination with the process A to provide controls for the amount of input nucleic acid, and to normalize data from tray to tray.

MethyLight™ Process C. The MethyLight™ process can be modified to avoid sequence discrimination at the PCR product detection level. Thus, in an additional qualitative process embodiment, just the primers are designed to cover CpG dinucleotides, and sequence discrimination occurs solely at the level of amplification. Preferably, the probe used in this embodiment is still a TaqMan® probe, but is designed so as not to overlap any CpG sequences present in the original, unmodified nucleic acid. The embodiment of process C represents a high-throughput, fluorescence-based real-time version of MSP technology, wherein a substantial improvement has been attained by reducing the time required for detection of methylated CpG sequences. Preferably, the reactions are monitored in real time by performing PCR amplification using 96-well optical trays and caps, and using a sequence detector (ABI Prism) to allow measurement of the fluorescent spectra of all 96 wells of the thermal cylcer continuously during the PCR amplification. Preferably, process C is run in combination with process A (below) to provide controls for the amount of input nucleic acid, and to normalize data from tray to tray.

MethyLight™ Process B. In preferred embodiments of the present invention, the MethyLight™ process can be also be modified to avoid sequence discrimination at the PCR amplification level. In a quantitative process B embodiment, just the probe is designed to cover CpG dinucleotides, and sequence discrimination occurs solely at the level of probe hybridization. Preferably, TaqMan® probes are used. In this version, sequence variants resulting from the bisulfite conversion step are amplified with equal efficiency; as long as there is no inherent amplification bias (Warnecke et al., Nucleic Acids Res. 25:4422-4426, 1997). Design of separate probes for each of the different sequence variants associated with a particular methylation pattern (e.g., 2³=8 probes in the case of 3 CpGs) would allow a quantitative determination of the relative prevalence of each sequence permutation in the mixed pool of PCR products. Preferably, the reactions are monitored in real time by performing PCR amplification using 96-well optical trays and caps, and using a sequence detector (ABI Prism) to allow measurement of the fluorescent spectra of all 96 wells of the thermal cylcer continuously during the PCR amplification. Preferably, process B is run in combination with process A, below to provide controls for the amount of input nucleic acid, and to normalize data from tray to tray.

MethyLight™ Process A. MethyLight™ process A does not, in itself, provide for methylated-CpG sequence discrimination at either the amplification or detection levels, but supports and validates the other three process applications by providing control reactions for the amount of input DNA, and to normalize data from tray to tray. Thus, if neither the primers, nor the probe overlie any CpG dinucleotides, then the reaction represents unbiased amplification and measurement of amplification using fluorescent-based quantitative real-time PCR serves as a control for the amount of input DNA. Preferably, process A not only lacks CpG dinucleotides in the primers and probe(s), but also does not contain any CpGs within the amplicon at all to avoid any differential effects of the bisulfite treatment on the amplification process. Preferably, the amplicon for process A is a region of DNA that is not frequently subject to copy number alterations, such as gene amplification or deletion.

Results obtained with the qualitative MethyLight™ version (process embodiment “B” of the technology) are described in the Examples below. Dozens of human tumor samples have been analyzed using this technology with excellent results.

Cancer Diagnostic and Prognostic Assays and Kits

Typically, diagnostic and/or prognostic assays of the present invention involve obtaining a tissue sample from a test tissue, performing a methylation assay on DNA derived from the tissue sample to determine the associated methylation state, and making a diagnosis or prognosis based thereon.

In preferred embodiments, diagnostic and prognostic cancer assays are based on determination of the methylation state of one or more of the disclosed 20 gene sequences (APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR, or methylation-altered DNA sequence embodiments thereof), as defined herein by the oligomeric primers and probes corresponding to SEQ ID NOS: 1-60, 64 and 65 (see TABLE II, below). SEQ ID NOS:61-63 correspond to the ACTB “control” gene region used in the present analysis (see EXAMPLE 1, below).

Additionally, other primers or probes corresponding to other sequence regions of the CpG islands associated with the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS sequence regions used herein may be used, based on the fact that the methylation state of a portion of a given CpG island is generally representative of the island as a whole.

Accordingly, the reagents required to perform one or more art-recognized methylation assays (including those described above) are combined with such primers and/or probes, or portions thereof, to determine the methylation state of CpG-containing nucleic acids.

For example, the MethyLight™, Ms-SNuPE, MCA, COBRA, and MSP methylation assays could be used alone or in combination, along with primers or probes comprising the sequences of SEQ ID NOS:1-65, or portions thereof, to determine the methylation state of a CpG dinucleotide within one or more of the 20 gene sequence regions corresponding to APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS or MTHFR, or, in the case of 19 of these 20 sequence regions (i.e., for all but MTHFR), to other CpG island sequences associated with these sequences, where such other CpG island sequences associated with these 19 gene sequences are those contiguous sequences of genomic DNA that encompasses at least one nucleotide of one of these 19 gene sequence regions, and satisfy the criteria of having both a frequency of CpG dinucleotides corresponding to an Observed/Expected Ratio>0.6, and a GC Content>0.5.

### EXAMPLE 1

CpG Island Hypermethylation Increased with the Progression of EAC

This Example shows the results of an analysis of the methylation status of a panel of CpG islands associated with 19 different genes selected for their known involvement in carcinogenesis or because they have been shown to be methylated in other tumors (see Table 1, and under “Definitions,” above), and of one non-CpG island sequence (MTHFR control sequence), for a total of 20 gene loci.

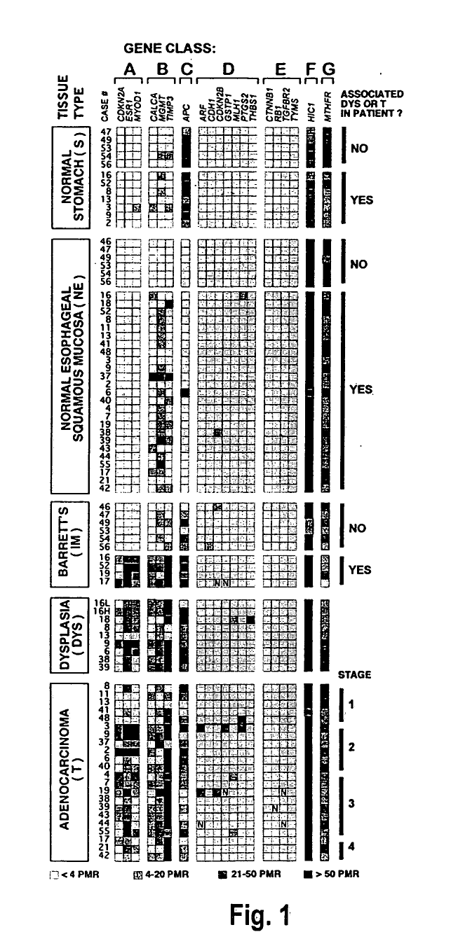

Quantitative methylation data of the 20 genes from a screen of 84 tissue specimens from 31 patients with different stages of Barrett's esophagus and/or associated adenocarcinoma showed a general increase in the frequency and in the quantitative level of CpG island hypermethylation at progressively advanced stages of disease. Accordingly, genes were grouped into distinct classes by their methylation behavior, based on both frequency and level of hypermethylation in various tissues (FIG. 1).

Materials and Methods

Sample Collection and histopathologic examination. Multiple tissue samples (normal esophagus (NE), normal stomach (S), intestinal metaplasia (IM), dysplasia (DYS) and/or adenocarcinoma (T)) from a total of 51 patients (range 39-86 years of age) with either adenocarcinoma or IM as the most advanced stage of disease were collected.

The initial set of samples analyzed included biopsies from 31 patients which were collected fresh and subdivided such that a part of each specimen was immediately frozen in liquid nitrogen and also embedded in paraffin for histopathologic examination by a pathologist (K.W.). Normal esophageal tissue was collected from every patient 10 cm or more away from the diseased areas. Frozen section examination of the frozen tissues was performed if the diagnosis was uncertain. The site of origin of the cancers was classified as esophageal if the epicenter of the tumor was above the anatomic gastroesophageal junction, with the junction defined as the proximal margin of the gastric rugal folds. TNM staging was used to classify the stage of each adenocarcinoma.

A second set of samples were obtained for a follow-up study of 20 cases. Two groups of IM samples were collected: patients that had only IM as the most advanced stage of disease (8 patients), and patients that had IM with associated dysplasia/adenocarcinoma located in another region of the esophagus (12 patients). H&E slides (5-micron sections) for each sample were prepared and examined by a pathologist (K.W.) to verify and localize the IM tissue. Cases that showed any signs of dysplasia or adenocarcinoma in the paraffm block used for analysis were excluded from this follow-up study. The IM tissues were carefully microdissected away from other cell types from a 30-micron section adjacent to the 5-micron H&E section. All specimens were classified according to the highest grade histopathologic lesion present in that sample. Approval for this study was obtained from the Institutional Review Board of the University of Southern California Keck School of Medicine.

Nucleic Acid Isolation. Genomic DNA was isolated from the frozen tissue biopsies by a simplified proteinase K digestion method (Laird et al., Nucleic Acids Res. 19:4293, 1991). The DNA from the paraffin tissues was extracted in lysis buffer (100 mM Tris-HCl, pH 8; 10 mM EDTA; and 1 mg/ml Proteinase K) overnight at 50° C. (Shibata et al., Am. J. Pathol. 141:539-543, 1992).

Sodium Bisulfite Conversion. Sodium bisulfite conversion of genomic DNA was performed as previously described (Olek et al., Nucleic Acids Res. 24:5064-5066, 1996). The beads were incubated for 14 hours at 50° C. to ensure complete conversion. Sodium bisulfite treatment converts unmethylated cytosines to uracil, while leaving methylated cytosine residues intact (Frommer et al., Proc. Natl. Acad. Sci. USA 89:1827-31, 1992).

MethyLight™ Analysis. After sodium bisulfite conversion, the methylation analysis was performed by the fluorescence-based, real-time PCR assay MethyLight™, as described herein, and as previously described (Eads et al., Cancer Res. 60:5021-5026, 2000; Eads et al., Cancer Res. 59:2302-2306, 1999; Eads et al., Nucleic Acids Res. 28:E32, 2000). Two sets of primers and probes, designed specifically for bisulfite converted DNA, were used: a methylated set for the gene of interest and a reference set, beta-actin (ACTB) to normalize for input DNA. Specificity of the reactions for methylated DNA were confirmed separately using human sperm DNA (with very low levels of CpG island methylation) and SssI (New England Biolabs)-treated sperm DNA (heavily methylated) as previously described (Eads et al., Cancer Res. 60:5021-5026, 2000).

The percentage of fully methylated molecules at a specific locus was calculated by dividing the GENE/ACTB ratio of a sample by the GENE/ACTB ratio of SssI-treated sperm DNA and multiplying by 100. The abbreviation PMR (Percent of Methylated Reference) is used to indicate this measurement. The methylation analysis on the paraffin microdissected samples was performed following bisulfite treatment as described above by an investigator blind to the associated dysplasia status of the samples.

TABLE II lists the MethyLight™ primer and probe sequences (SEQ ID NOs:1-65), based on Genbank sequence data (except for SEQ ID NOs:64 and 65, see below), used in the present methylation analysis. Three oligos were used in every reaction: two locus-specific PCR primers flanking an oligonucleotide probe with a 5′ fluorescent reporter dye (6FAM) and a 3′ quencher dye (TAMRA) (Livak et al., PCR Methods Appl. 4:357-362, 1995). The Genbank accession number for each sequence is listed with the corresponding PCR amplicon location within that sequence. The % GC content, CpG observed/expected value and CpG:GpC ratio of 200 base pairs encompassing the MethyLight amplicon are indicated for each gene. The reaction type is designated “M” for methylation reaction and “C” for control reaction. The bisulfite treated DNA strand (top (“T”) or bottom (“B”)) and amplicon orientation (parallel (“P”) or antiparallel (“A”)) is also indicated. All primer and probe sequences are listed in the 5′ to 3′ direction. The numbers in brackets after each primer or probe sequence correspond to the associated SEQ ID NOs. The single asterisk (*) notes that there are two bases in our CDKN2A primers that differ from this GenBank sequence, since a preliminary high-throughput GenBank entry was the only available sequence at the time of applicants' primer design. The correct primers should be the following: forward, TGGAGTTTTCGGTTGATTGGTT (SEQ ID NO:64) and reverse, AACAACGCCCGCACCTCCT (SEQ ID NO:65). The bases differing from the GenBank sequences are underlined. The double asterisk (**) indicates that the start site is not well defined.

TABLE II

MethyLight ™ primer and probe sequences

| | | | | | | | | | | |
|---|---|---|---|---|---|---|---|---|---|---|
| | | | | Amplicon | | | | | | |
| | Gen- | | Amplicon | Location | | | | | | |
| | bank | Bisul- | Location | (bp) Rela- | | | | | | |
| HUGO | Acces- | fite | (Genbank | tive to | % GC | Obs/ | Forward | | Reverse | |
| Gene | sion | (T or B | Num- | Transcrip- | Con- | Exp | | Forward | Primer | Primer |
| Name | Number | Strand) | bering) | tion Start | tent | CpG | CpG:GpC | Sequence (5′-3′) | Probe Sequence (5′-3′) | Sequence (5′-3′) |

“Class A” genes‡

| | | | | | | | | | | |
|---|---|---|---|---|---|---|---|---|---|---|
| CDKN2A | NM | T | 66- | +9/ | 72 | 1.68 | 0.81 | TGGAATTTTCG | 6FAM- | AACAACGTCCG |
| * | _000 | | 133bp | +69 | | | | GTTGATTGGTT | ACCCGACCCCGAACCGC | CACCTCCT [2] |
| | 077 | | | | | | | [1] | G-TAMRA [3] | |
| ESR1 | X62 | T | 2784bp- | +14/ | 69 | 1.52 | 0.83 | GGCGTTCGTTT | 6FAM- | GCCGACACGCG |
| | 462 | | 2884bp | +114 | | | | TGGGATTTG [4] | CGATAAAACCGAACGAC | AACTCTAA [5] |
| | | | | | | | | | CCGACGA-TAMRA [6] | |
| MYOD1 | AF0 | T | 9889bp- | −375/ | 61 | 1.12 | 0.74 | GAGCGCGCGT | 6FAM-CTCCAACACCCGA | TCCGACACGCC |
| | 2714 | | 9962bp | −302 | | | | AGTTAGCG [7] | CTACTATATCCGCGAAA- | CTTTCC [8] |
| | 8 | | | | | | | | TAMRA [9] | |

“Class B” genes

| | | | | | | | | | | |
|---|---|---|---|---|---|---|---|---|---|---|
| CALCA | X15 | T | 1706bp- | −128/ | 60 | 0.72 | 0.53 | GTTTTGGAAGT | 6FAM-ATTCCGCCAATAC | TTCCCGCCGCTA |
| | 943 | | 1806bp | −28 | | | | ATGAGGGTGAC | ACAACAACCAATAAACG- | TAAATCG [11] |
| | | | | | | | | G [10] | TAMRA [12] | |
| MGMT | U95 | B | 206bp- | +392/ | 67 | 0.88 | 0.55 | CTAACGTATAA | 6FAM-CCTTACCTCTAAAT | AGTATGAAGGG |
| | 038 | | 297bp | +483 | | | | CGAAAATCGTA | ACCAACCCCAAACCCG- | TAGGAAGAATT |
| | | | | | | | | ACAACC [16] | TAMRA [18] | CGG [17] |
| TIMP3 | U33 | T | 1051bp- | +1051/ | 78 | 1.04 | 0.63 | GCGTCGGAGGT | 6FAM- | CTCTCCAAAATT |
| | 110 | | 1143 | +1143 | | | | TAAGGTTGTT | AACTCGCTCGCCCGCCGA | ACCGTACGCG |
| | | | | | | | | [13] | A-TAMRA [15] | [14] |

“Class C” genes

| | | | | | | | | | | |
|---|---|---|---|---|---|---|---|---|---|---|
| APC | U02 | B | 759bp- | +7/ | 69 | 1.36 | 0.71 | GAACCAAAACG | 6FAM- | TTATATGTCGGT |
| | 509 | | 832bp | +80 | | | | CTCCCCAT [19] | CCCGTCGAAAACCCGCC | TACGTGCGTTTA |
| | | | | | | | | | GATTA-TAMRA [21] | TAT [20] |

“Class D” genes

| | | | | | | | | | | |
|---|---|---|---|---|---|---|---|---|---|---|
| ARF | AF0 | T | 5447bp- | −203/ | 78 | 2 | 0.89 | ACGGGCGTTTT | 6FAM- | CCGAACCTCCA |
| | 8233 | | 5515bp | −135 | | | | CGGTAGTT [22] | CGACTCTAAACCCTACGC | AAATCTCGA [23] |
| | 8 | | | | | | | | ACGCGAAA-TAMRA [24] | |
| CDH1 | L34 | T | 842bp- | −180/ | 69 | 1.2 | 0.68 | AATTTTAGTT | 6FAM- | TCCCCAAAACG |
| | 545 | | 911bp | −111 | | | | AGAGGGTTATC | CGCCCACCCGACCTCGCA | AAACTAACGAC |
| | | | | | | | | GCGT [25] | T-TAMRA [27] | [26] |
| CDKN2B | S757 | T | 350bp- | +222/ | 64 | 1.44 | 0.82 | AGGAAGGAGAG | 6FAM- | CGAATAATCCA |
| | 56 | | 430bp | +302 | | | | AGTGCGTCG | TTAACGACACTCTTCCCTT | CCGTTAACCG |
| | | | | | | | | [28] | CTTTCCCACG-TAMRA [30] | [29] |
| GSTP1 | M24 | T | 1146bp- | −79/ | 75 | 2 | 0.93 | GTCGGCGTCGT | 6FAM-AAACCTCGCGACC | AAACTACGACG |
| | 485 | | 1245bp | +21 | | | | GATTTAGTATT | TCCGAACCTTATAAAA- | ACGAAACTCCA |
| | | | | | | | | G [31] | TAMRA [33] | A [32] |
| MLH1 | U26 | T | 254bp- | −662/ | 68 | 2.08 | 1.04 | CGTTATATATC | 6FAM- | CTATCGCCGCCT |
| | 559 | | 341bp | −575 | | | | GTTCGTAGTAT | CGCGACGTCAAACGCCA | CATCGT [34] |
| | | | | | | | | TCGTGTTT [35] | CTACG-TAMRA [36] | |
| PTGS2 | AF0 | T | 6779bp- | −362/ | 62 | 0.8 | 0.71 | CGGAAGCGTTC | 6FAM- | AATTCCACCGCC |
| | 4420 | | 6924bp | −217 | | | | GGGTAAAG [37] | TTTCCGCCAAATATCTTTT | CCAAAC [38] |
| | 6 | | | | | | | | CTTCTTCGCA-TAMRA [39] | |
| THBS1 | J048 | B | 1642bp- | −636/ | 75 | 2.08 | 1 | CGACGCACCAA | 6FAM- | GTTTTGAGTTGG |
| | 35 | | 1716bp | −562 | | | | CCTACCG [40] | ACGCCGCGCTCACCTCCC | TTTTACGTTCGT |
| | | | | | | | | | T-TAMRA [42] | T [41] |

“Class E” genes

| | | | | | | | | | | |
|---|---|---|---|---|---|---|---|---|---|---|
| CTNNB1 | X89 | T | 583bp- | −413/ | 75 | 1.92 | 1 | GGAAAGGCGC | 6FAM- | TCCCCTATCCCA |
| | 448 | | 664bp | −332 | | | | GTCGAGT [43] | CGCGCGTTTCCCGAACCG- | AACCCG [44] |
| | | | | | | | | | TAMRA [45] | |
| RB1 | L11 | T | 1781bp- | −279/ | 68 | 2.24 | 1.27 | TTAGTTCGCGT | 6FAM- | ACTAAACGCCG |
| | 910 | | 1900bp | −160 | | | | ATCGATTAGCG | TCACGTCCGCGAAACTCC | CGTCCAA [47] |
| | | | | | | | | [46] | CGA-TAMRA [48] | |
| TGFBR2 | U52 | T | 256bp- | −58/ | 75 | 2.08 | 0.81 | GCGCGGAGCG | 6FAM- | CAAACCCCGCT |
| | 240 | | 333bp | +19 | | | | TAGTTAGG [49] | CACGAACGACGCCTTCCC | ACTCGTCAT [50] |
| | | | | | | | | | GAA-TAMRA [51] | |
| TYMS | D00 | T | 1052bp- | +301/ | 75 | 2.16 | 0.9 | CGGCGTTAGGA | 6FAM-CCGAATACCGACA | TCTCAAACTATA |
| | 517 | | 1128bp | +377 | | | | AGGACGAT [52] | AAATACCGATACCCGT- | ACGCGCCTACA |
| | | | | | | | | | TAMRA [54] | T [53] |

“Class F” genes

| | | | | | | | | | | |
|---|---|---|---|---|---|---|---|---|---|---|
| HIC-1 | L41 | T | 562bp- | −55/ | 71 | 1.12 | 0.5 | GTTAGGCGGTT | 6FAM-CAACATCGTCTAC | CCGAACGCCTC |
| | 919 | | 662bp | +45** | | | | AGGGCGTC [58] | CCAACACACTCTCCTACG- | CATCGTAT [59] |
| | | | | | | | | | TAMRA [60] | |

“Class G” genes

| | | | | | | | | | | |
|---|---|---|---|---|---|---|---|---|---|---|
| MTHFR | AF1 | T | 123bp- | +72/ | 57 | 0.56 | 0.54 | TGGTAGTGAGA | 6FAM- | CGCCTCATCTTC |
| | 0597 | | 212bp | +45 | | | | GTTTTAAAGAT | TCTCATACCGCTCAAAAT | TCCCGA [56] |
| | 7 | | | | | | | AGTTCGA [55] | CCAAACCCG-TAMRA [57] | |

“Control” gene

| | | | | | | | | | | |
|---|---|---|---|---|---|---|---|---|---|---|
| ACTB | Y00 | T | 390bp- | −1,599/ | 58 | 0.16 | 0.14 | TGGTGATGGAG | 6FAM-ACCACCACCCAAC | AACCAATAAAA |
| | 474 | | 522bp | −1,467** | | | | GAGGTTTAGTA | ACACAATAACAAACACA- | CCTACTCCTCCC |
| | | | | | | | | AGT [61] | TAMRA [63] | TTAA 62] |

TABLE II cont. MethyLight ™ primer and probe sequences. Numbers in brackets correspond to SEQ ID NOs: 1-63.
{Gene “Classes” are defined according to the present invention.

Statistics. The PMR values obtained by MethyLight™ (see above) were “dichotomized” at 4 PMR for statistical purposes as described previously (Eads et al., Cancer Res. 60:5021-5026, 2000. Dichotomization facilitates graphical representation, and moderates the quantitative impact of gene loci with different levels of hypermethylation, resulting in a more reliable cross-gene comparison of hypermethylation frequencies. Specifically, dichotomization equalizes the quantitative impact of methylated genes within each class (see “Epigenetic gene classes,” below), simplifying cross-gene comparisons of methylation frequencies.

A dichotomization point of 4 PMR was selected because it gave the best discrimination between normal and malignant tissues, across the board for all CpG islands (Eads et al., Cancer Res. 60:5021-5026, 2000). However, the precise dichotomization point does not significantly affect the statistics or alter the conclusions, and other dichotomization points are within the scope of the present invention (see below).

Accordingly, samples containing 4 PMR or higher were designated as methylated and given a value of 1, while samples containing less than 4 PMR were designated as unmethylated and given a value of 0. The cumulative value of genes methylated in each class (see Epigentic gene classes” A-G, herein below), or for all 19 genes was then used as a continuous variable in a Fisher's Protected Least Significant Difference test, adapted for use with unequal sample sizes (SAS Statview software) to obtain p-values. The different parameters such as tissue type, presence of associated dysplasia, tumor stage, etc., were used as the nominal variables. The IM samples in the above-mentioned “follow-up” study of hypermethylation in IM, and the presence of associated dysplasia and/or carcinoma, were further dichotomized at 1 or fewer, versus two or more Class A genes methylated. A Fisher's exact test was then used to determine statistical significance.

Results

CpG Island Hypermethylation and the Progression of EAC. The methylation status of a panel of CpG islands associated with 19 different genes and of one non-CpG island sequence for a total of 20 gene loci, was analyzed by the quantitative, high-throughput MethyLight™ assay (Eads et al., Cancer Res. 59:2302-2306, 1999; Eads et al., Nucleic Acids Res. 28:E32, 2000). The efficiencies of the methylation reactions were controlled for in each analysis by including unmethylated control DNA and methylated control DNA (Eads et al., Cancer Res. 60:5021-5026, 2000). The 20 genes were selected for their known involvement in carcinogenesis or because they have been shown to be methylated in other tumors (see Table 1, and under “Definitions,” above). We included a region located in the MTHFR gene as a “non-CpG island” control for a single copy sequence that does not satisfy the criteria (see “Definitions,” above) of a CpG island. CpG dinucleotides outside of an island are presumably normally methylated, unlike CpG dinucleotides within CpG islands.

FIG. 1 illustrates the quantitative methylation data of the 20 genes from our screen of 84 tissue specimens from 31 patients with different stages of Barrett's esophagus and/or associated adenocarcinoma. Methylation analysis was performed using the MethyLight assay (Eads et al., Cancer Res. 59:2302-2306, 1999; Eads et al., Nucleic Acids Res. 28:E32, 2000). The percentage of fully methylated molecules at a specific locus (PMR=Percent of Methylated Reference) was calculated by dividing the GENE/ACTB ratio of a sample by the GENE/A CTB ratio of SssI-treated sperm DNA and multipling by 100. The resulting percentages were then dichotomized at 4% PMR to facilitate graphical representation and to reveal tissue-specific patterns. The various squares, each having one of four possible shading intensity levels (see bottom axis of FIG. 1), designate samples with less than 4 PMR, 4-20 PMR, 21-50 PMR and more than 51 PMR, where progressively increasing shading intensity levels correspond to progressively higher PMR values. The tissue types are shown on the left. The TNM tumor staging is designated by “1”, “2”, “3” and “4”. The occurrence of distally located dysplasia and/or adenocarcinoma in the patient is indicated at the right of the figure by “YES” if present and “NO” if absent. ‘W’ indicates an analysis for which the control gene ACTB did not reach sufficient levels to allow the detection of a minimal value of 1 PMR for that methylation reaction in that particular sample.

There was a general increase in the frequency and in the quantitative level of CpG island hypermethylation at progressively advanced stages of disease. However, the propensity for aberrant methylation of the genes was not uniform. Genes differed both in their frequency and in their levels of hypermethylation in various tissues.

Therefore, according to the present invention, genes can be grouped into classes based on their methylation behavior (Classes A-G, as shown at the right of FIG. 1). This allowed for a visual assessment of concordant methylation of the different genes during various stages of turmorigenesis. A rationale for each of the gene classes is presented in the following section.

Epigenetic Gene Classes. The analysis of combined behavior of genes with different levels of DNA methylation would, without appropriate data treatment, be expected to lead to a bias of the group behavior towards genes with quantitatively high levels of DNA methylation. For instance, the mean values for gene “Class B” for most of the tumor samples would be driven primarily by the TIMP3 values, since this gene tended to have higher levels of methylation than the other two genes in this group (see FIG. 1).

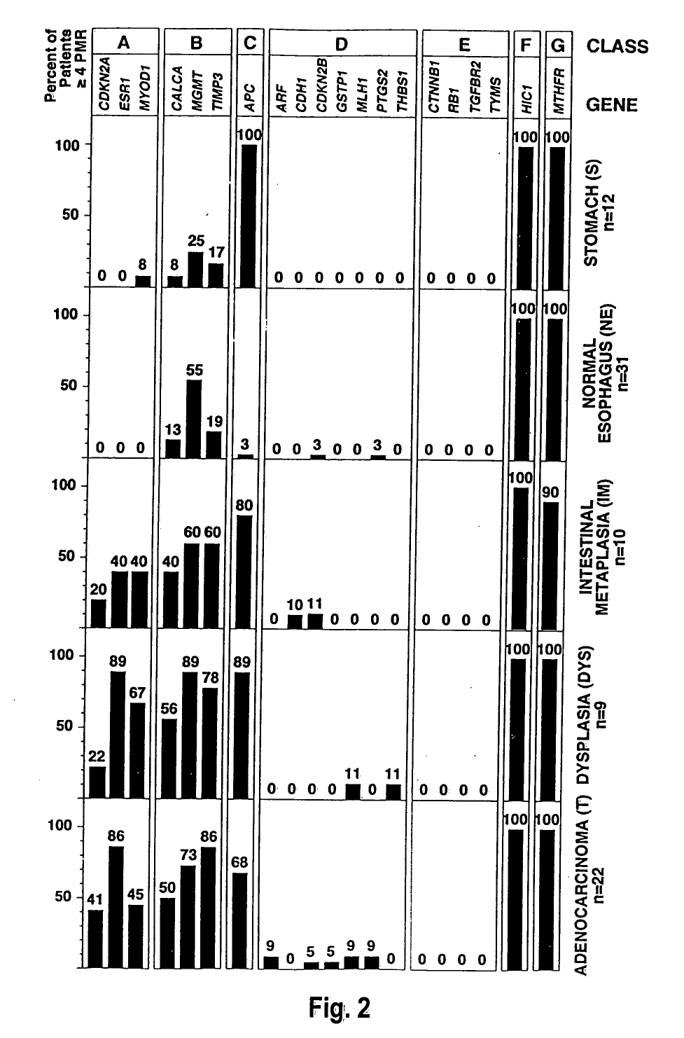

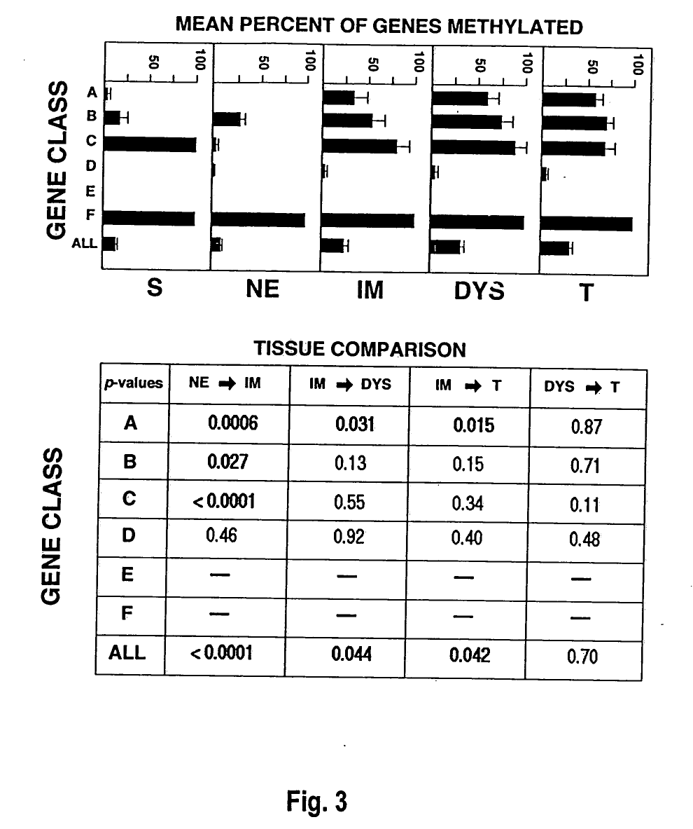

Therefore, the methylation values used to generate FIG. 1 were collapsed into a binary variable with a dichotomization point of 4 PMR to equalize the quantitative impact of methylated genes within each epigenetic class. Samples containing 4 PMR or higher were designated as methylated and given a value of 1, while samples containing less than 4 PMR were designated as unmethylated and given a value of 0 (see “Statistics” above, under “Materials and Methods”). This dichotomization moderates the effect of highly methylated genes, simplifies cross-gene comparisons of methylation frequencies, as shown in FIG. 2, and allowed the calculation of class averages of methylation frequencies as shown in FIG. 3 (below).

FIG. 2 shows the percent of samples methylated for each gene by tissue type. The data was dichotomized at 4 PMR, with 4 PMR and higher designated as methylated, and below 4 PMR as unmethylated. The genes, according to the present invention, were grouped according to their respective epigenetic gene classes (A-G) as shown in FIG. 1. The letter “n” equals the number of samples analyzed for each tissue.

The suitability of the 4 PMR dichotomization point was based on its ability to discriminate between the different tissue types, as shown in FIGS. 1-3 (see also Klump et al., Gastroenterology. 115:1381-1386, 1998). Other dichotomization point values are within the scope of the present invention, where such dichotomization point values moderate the statistical effects of highly methylated genes, simplify cross-gene comparisons of methylation frequencies, and facilitate calculation of class averages of methylation frequencies. For instance, there is still a statistically significant difference in the mean percent of genes methylated (out of 19 genes) between the normal esophageal mucosa and the IM (p=0.0003), DYS (p<0.0001) and T (p<0.0001) tissues when the data is dichotomized at 10 PMR.

Additionally, all of the statistically significant findings of the NE and IM methylation frequency with or without associated dysplasia (see Example 3, below) remain significant at a dichotomization point of 10 PMR, instead of 4 PMR. It is important to note that 4 PMR is not comparable to a 4% methylation level of a single CpG dinucleotide. Rather, it indicates that in this sample, 4% of the DNA molecules had complete methylation at all CpG dinucleotides covered by the three MethyLight™ primers (usually about 8 CpGs). The nature of the MethyLight™ assay is such that it is oblivious to all other methylation patterns that may be present (Eads et al., Nucleic Acids Res. 28:E32, 2000).

Therefore, 4 PMR is likely to represent a higher mean level of methylation than 4%. The extensively methylated molecules that are assayed by MethyLight™ are likely to represent alleles that have been completely silenced by CpG island hypermethylation, although this was not investigated herein.

Of the panel of 20 genes, the most informative genes were those with an intermediate frequency of hypermethylation (ranging from 15% (CDKN2A) to 60% (MGMT) of the sample values above the 4 PMR methylation cutoff). This group was further subdivided into three epigenetic gene classes according to the absence (Class “A”) or presence (Class “B”) of methylation in normal esophageal mucosa and stomach, or the infrequent methylation of normal esophageal mucosa accompanied by methylation in all normal stomach samples (Class “C”). The other genes were less informative, since the incidence of hypermethylation was either very infrequent (Class “D”), completely absent (Class “E”), or ubiquitous (Classes “F” and “G”) regardless of tissue type (FIGS. 1, 2 and 3).

Epigenetic gene Class A comprises the genes CDKN2A, ESR1 and MYOD1 (FIGS. 1, 2 and 3). There was a statistically significant difference in the methylation frequency of ESR1 (p=0.0001) and MYOD1 (p=0.0038) of normal esophagus (NE), as compared to IM tissue, but not for CDKN2A (p=0.097). The frequency of CDKN2A methylation increased significantly in the more advanced stages of the adenocarcinoma (T) (p<0.0001).

Epigenetic gene Class B comprises the genes CALCA, MGMTand TIMP3. In contrast to Class A, this class exhibited methylation in the normal esophageal mucosa (NE) and stomach (S) tissue (FIGS. 1 and 2). Only TIMP3 showed a significant difference in methylation frequency between the NE and IM values (p=0.0074).

Epigenetic gene Class C comprises the gene APC which was, in contrast to genes of Classes A and B, methylated in all normal stomach samples (FIGS. 1 and 2). This confirms previous documentation of APC methylation in normal stomach tissue (Eads et al., Cancer Res. 60:5021-5026, 2000). The mechanism which protects APC from methylation in the normal esophageal tissues (NE) but not in normal stomach tissues (S) is not clear.

Epigenetic gene Class D comprises the genes ARF, CDH1, CDKN2B, GSTP1, MLH1, PTGS2 and THBS1, which were infrequently methylated (FIGS. 1 and 2). There was a slight increase in the frequency of this class of genes in adenocarcinoma (T), but this did not approach statistical significance (FIG. 3). Interestingly, with the exception of PTGS2, which has not yet been investigated in other systems, the remaining Class D genes are frequently hypermethylated in other tumor types (Table 2).

Epigenetic gene Class E comprises the CTNNB1, RB1, TGFBR2 and TYMS1 genes, which were unmethylated at each stage in the progression of EAC. Similar to most Class D genes, RB1 and TGFBR2 have been found to be hypermethylated in other tumors types (see Table 1, and literature references under “DEFINITIONS” herein above). It should be noted that all samples scored postitive for DNA input as measured by the control gene (ACTB). Therefore, the lack of detectable DNA methylation cannot be attributed to a lack of input DNA. The control reaction was sufficient in each sample, so that a level as low as 1 PMR for a given test gene could be detected. The integrity and specificity of all methylation reactions was confirmed using in vitro methylated human DNA.

The epigenetic Class F comprises the HIC1 gene, which was completely methylated, regardless of tissue type (FIGS. 1 and 2). HIC1 is commonly methylated in other types of cancers (Jones & Laird, Nat. Genet. 21:163-167, 1999; Baylin & Herman, Trends Genet. 16:168-174, 2000), and has been shown to be methylated in normal breast ductal tissue and bone marrow samples of breast cancer and AML patients, respectively (Melki et al., Cancer Res. 59:3730-3740, 1999; Fujii et al., Oncogene. 16:2159-2164, 1998). Nevertheless, the finding of ubiquitous methylation of a CpG island in normal tissues was unexpected. Therefore, the validity of the HIC1 MethyLight™ results was confirmed using a different technique (HpaII-PCR) (Singer-Sam et al., Nucleic Acids Res. 18:687, 1990).

Epigenetic Class G comprises the non-CpG island MTHFR gene, used herein as a control. Interestingly, the ubiquitous HIC1 methylation pattern is similar to the non-CpG island MTHFR control (Class G), however the percentage of methylated molecules was quantitatively higher for HIC1 (FIG. 1).

Epigenetic Profiles ofEAC Progression. Each tissue type showed a unique epigenetic profile or fingerprint that changed during disease progression (FIG. 3, upper panel).

FIG. 3 shows a comparison of epigenetic profiles according-to the present invention. The data was dichotomized at 4 PMR, with 4 PMR and higher designated as methylated, and below 4 PMR as unmethylated. Error bars represent the standard error of the mean. Upper panel: Mean percent of genes methylated in each gene Class (A-F or ALL 19 CpG islands) by tissue type (N, normal esophagus; S, stomach; IM, intestinal metaplasia; DYS, dysplasia; T, adenocarcinoma). The error bars represent the standard error of the mean (SEM). Lower panel: Statistical analysis of the difference in mean percent of genes methylated in different tissues by gene Class (A-F) or for all 19 CpG islands combined (ALL). The p-values were generated by a Fisher's Protected Least Significant Difference (PLSD) test, adapted for use with unequal sample numbers (SAS Statview™ software).

Classes A, B and C were methylated at a significantly higher frequency in IM tissue than in normal esophageal mucosa (NE) (FIG. 3, upper and lower panels). Furthermore, the transition from IM to dysplasia (DYS) or malignancy (T) was associated with an additional increase in Class A methylation (FIG. 3, upper and lower panels). The lack of a significant difference between dysplasia and adenocarcinoma for any of the gene classes or when all 19 genes are combined (FIG. 3, upper and lower panels) suggests that most of these abnormal epigenetic alterations occur early in the progression of EAC.

In summary of this Example. According to the present invention, quantitative methylation data of 20 genes (Tables I and II, above) from a screen of 84 tissue specimens from 31 patients with different stages of Barrett's esophagus and/or associated adenocarcinoma showed a general increase in the frequency and in the quantitative level of CpG island hypermethylation at progressively advanced stages of disease (FIGS. 1-3, above).

Additionally, genes were grouped into novel epigenetic classes based on their methylation behavior (Classes A-G, as shown herein in FIGS. 1-3) during tumor progression. This allowed for graphical representation of concordant methylation of the different genes during various stages of turmorigenesis, which can be readily appreciated by means of a simple visual assessment.

Each tissue type showed a unique epigenetic profile or fingerprint that changed during disease progression (FIG. 3, upper panel). Classes A, B and C were methylated at a significantly higher frequency in IM tissue than in normal esophageal mucosa (NE) (FIG. 3, upper and lower panels). Furthermore, the transition from IM to dysplasia (DYS) or malignancy (T) was associated with an additional increase in Class A methylation (FIG. 3, upper and lower panels).

### EXAMPLE 2

Hypermethylation was Reflective of EAC Tumor Grade and Stage

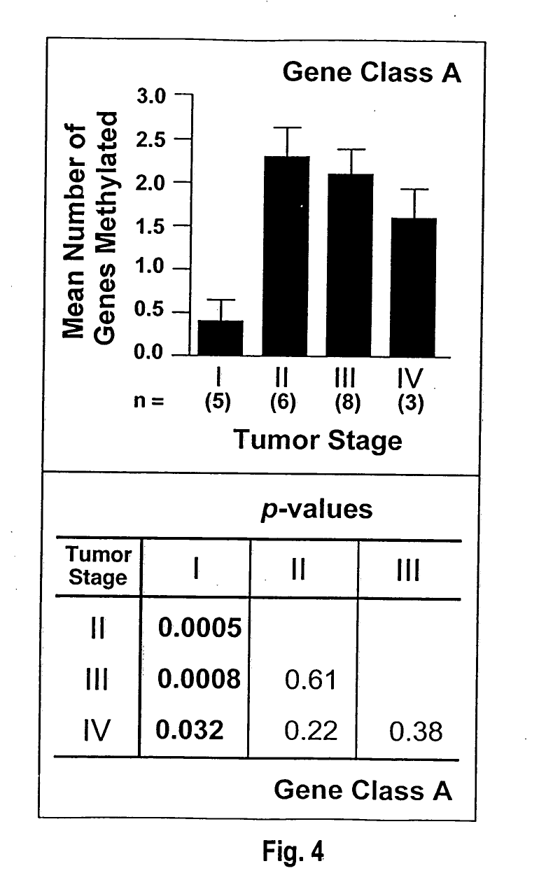

This Example examines whether the grade or stage of an esophageal adenocarcinoma correlates with a higher frequency of CpG island hypermethylation. According to the present invention, for EAC, epigenetic Class A gene methylation is significantly higher in stage II, III and IV tumors relative to less advanced stage I tumors (FIG. 4).

Materials and Methods

TNM staging. The American Joint Committee on Cancer (“AJCC”) has designated staging by TNM classification (Tumor; lymph Node metastasis, distant Metastasis). TNM staging was used to classify the stage of each esophageal adenocarcinoma from the tissues of Example 1.

Methylation and statistical analysis. Methylation and statistical analysis was as described herein under Example 1.

Results

Methylation of epigenetic Class A genes increases with tumor stage. Moderately differentiated tumors have significantly less frequent Class A methylation compared to poorly differentiated tumors (p=0.045). Additionally, FIG. 4 (upper and lower panels) shows that there is a significantly higher mean number of Class A genes methylated in stage II, III and IV tumors relative to less advanced stage I tumors. The differences between stage I tumors and stage II, III and IV tumors did not reach statistical significance for any of the other epigenetic gene classes.

FIG. 4 shows the relationship between Class A methylation frequency and tumor stage according to the present invention. The data was dichotomized at 4 PMR, with 4 PMR and higher designated as methylated, and below 4 PMR as unmethylated. Upper panel: Mean number of genes methylated for Class A with respect to tumor stage (I-IV) is shown (see FIG. 1). The error bars represent the standard error of the mean (SEM). The letter “n” equals the number of samples analyzed in each tumor stage. Lower panel: Statistical analysis of the difference in mean number of Class A genes methylated by tumor stage. The p-values were generated by a Fisher's Protected Least Significant Difference (PLSD) test, adapted for use with unequal sample numbers (SAS Statview™ software).

In summary for this Example. According to the present invention, in addition to the epigenetic profiles or fingerprints (comprising the gene classes disclosed herein) that can be used to assess oncogenic progression, the mean number of methylated Class A genes can be used to assess the relative stages of EAC tumors.

### EXAMPLE 3

Methylation of Premalignant Tissues With or Without Associated Dysplasia

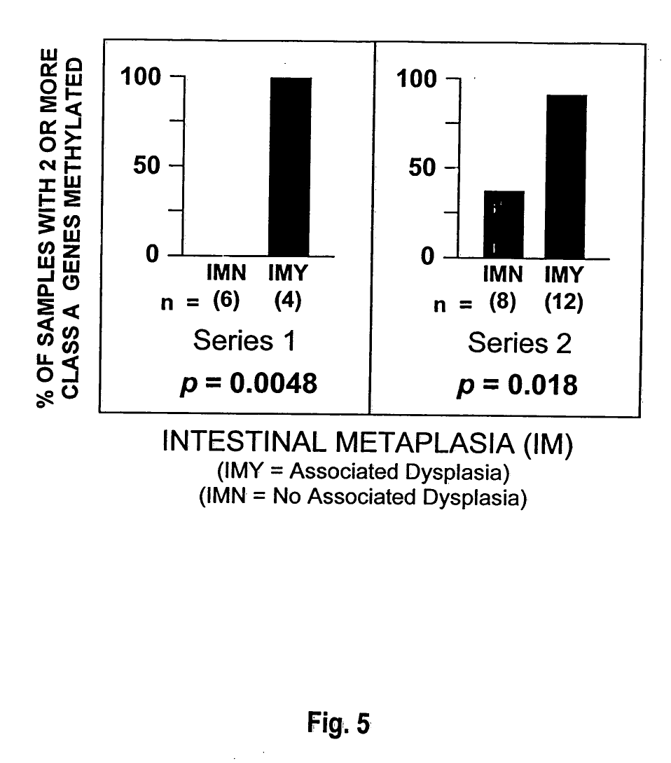

This Example shows that the frequency of Class B methylation in the normal esophagus (NE) was found to be significantly higher in patients with associated dysplasia/tumor (p=0.0037) (FIG. 1). Additionally, Class A methylation was found to be more frequent in IM samples from patients with concurrent dysplasia or cancer, than in IM samples from patients without any evidence of further progression (p<0.0001) (FIGS. 1 and 5). That is, there was a significant positive association between hypermethylation of epigenetic Class A genes in IM tissue, and the presence of associated dysplasia or cancer (FIG. 5).

Materials and Methods

Histopathology. Histopathological classification was as described under “Materials and Methods,” Example I above.

Methylation and statistical analysis. Methylation and statistical analysis was as described herein under Example 1.

Results

Methylation of Premalignant Tissues with or without Associated Dysplasia. The occurrence, according to the present ivention, of CpG island hypermethylation in some cases of IM for Class A and some cases of normal esophageal mucosa for Class B raised the question whether these methylation events represent normal methylation patterns in these non-dysplastic tissues, or whether they reflect methylation changes that predispose cells to further progression. In the latter case, one would expect to find a higher frequency of such CpG island hypermethylation in these tissues in patients who have already undergone further disease progression. Therefore, the frequency of such CpG island hypermethylation was compared between tissues (of the present study) with or without associated dysplasia.

In the initial study, patients were divided based on whetier or not they had Barrett's esophagus (IM) as their most advanced stage of disease (FIG. 1, “NO”) or whether they had associated dysplasia and/or adenocarcinoma present in a different region of the esophagus (FIG. 1, “YES”). The frequency of Class B methylation in the normal esophagus (NE) was indeed found to be significantly higher in patients with associated dysplasia/tumor (p=0.0037) (FIG. 1). Additionally, Class A methylation was found to be more frequent in IM samples from patients with concurrent dysplasia or cancer, than in IM samples from patients without any evidence of further progression (p<0.0001) (FIG. 1).

A potential criticism of this analysis is that the same set of samples was used to delineate the class of genes, as was used to test the association with a clinical parameter. Therefore, a follow-up study of 20 additional cases of IM was performed entirely independent of the first data set.

In the follow-up study of 20 cases, two groups of IM samples were collected: patients that had only IM as the most advanced stage of disease (8 patients), and patients that had IM with associated dysplasia/adenocarcinoma located in another region of the esophagus (12 patients). H&E slides (5-micron sections) for each sample were prepared and examined by a pathologist (K.W.) to verify and localize the IM tissue. Cases that showed any signs of dysplasia or adenocarcinoma in the paraffin block used for analysis were excluded from this follow-up study. The IM tissues were carefully microdissected away from other cell types from a 30-micron section adjacent to the 5-micron H&E section. All specimens were classified according to the highest grade histopathologic lesion present in that sample.

The initial study had revealed that all IM samples associated with further disease progression (“YES”) had at least two Class A genes methylated, while all IM samples without associated dysplasia or adenocarcinoma (“NO”) did not show any methylation of Class A genes (FIG. 1, under “Barrett's (IM)”). Therefore, a state of having two or more Class A genes methylated was defined as an indicator of increased risk for the presence of associated dysplasia or cancer.

The data from our first series gave a p-value of 0.0048 in a Fisher's exact test of this association (FIG. 5, left panel). The follow-up series of 20 independent cases gave a p-value of 0.018 (FIG. 5, right panel).

FIG. 5 shows the percent of two or more Class A genes methylated in intestinal metaplasia (“IM”) tissues with (“Y”), or without (‘N’) associated dysplasia and/or adenocarcinoma. The data was dichotomized at 4 PMR, with 4 PMR and higher designated as methylated, and below 4 PMR as unmethylated. Left panel: Class A methylation in the IM data illustrated in FIG. 1. Right panel: Class A methylation in the IM for a completely independent follow-up study of twenty different microdissected IM samples. The error bars represent the standard error of the mean (SEM). The letter “n” equals the number of samples analyzed in each tissue group.

Therefore, the positive association between hypermethylation of Class A genes and the presence of associated dysplasia or cancer is significant. It should be noted that the IM samples without associated dysplasia in this follow-up study (FIG. 5, right panel) showed a low frequency of samples with at least two genes methylated, which is in contrast to the absence of methylation in the first study (FIG. 1, and FIG. 5, left panel). This may be attributed to the fact that the samples in the second series were microdissected from paraffin sections. Therefore, there is a lower background of unmethylated stromal cells in the sample. In this case, the methylation signal is not as diluted by other normal cells and consequently the ratio of methylated molecules to total DNA may rise above the 4 PMR threshold. Alternatively, dysplastic or malignant tissue may have been missed during the endoscopic survey in some of the cases scored as free of further disease progression due to the sampling limitations of endoscopy. This is a well-documented problem in the detection of esophageal adenocarcinoma (Peters et al., J. Thorac. Cardiovasc. Surg. 108:813-821, 1994).

### EXAMPLE 4

No Clear Evidence of CpG Island Methylator Phenotype (“CIMP”) for EAC

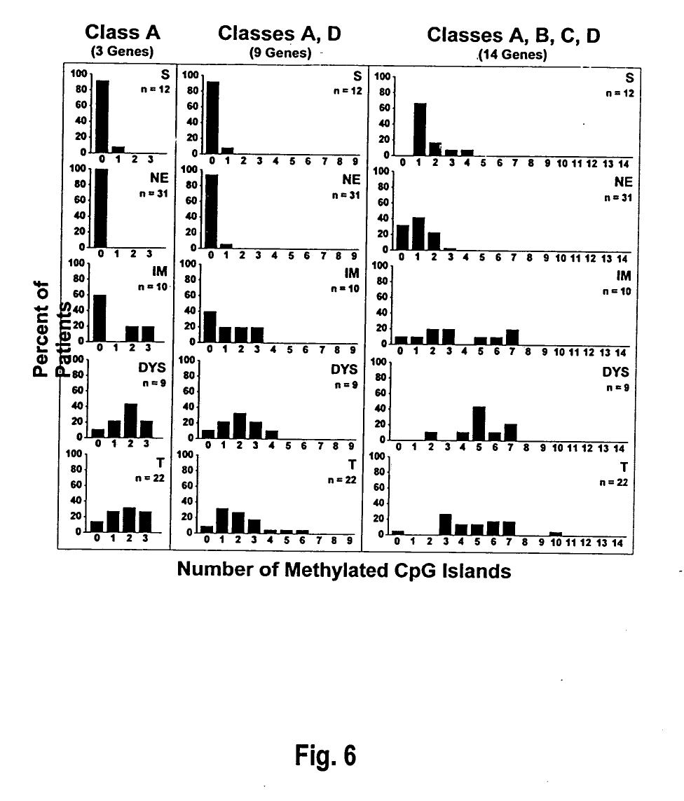

This Example shows that, for the present study of EAC, there was no clear evidence of a separate group of CIMP tumors, as has been previously defined for colorectal and gastric cancer (Toyota et al., Proc. Natl. Acad. Sci. USA. 96:8681-8686, 1999; Toyota et al., Cancer Res. 59:5438-5442, 1999). However, CpG island hypermethylation in EAC did occur across multiple loci in a given sample. Furthermore, the number of loci hypermethylated in a single sample increased as the disease progressed through different histological stages (FIG. 6). The bimodal distributions seen in IM tissues (FIG. 6) can be fully attributed to the concurrent association with dysplasia or cancer described herein above.

Materials and Methods

Histopathology. Histopathological classification was as described under “Materials and Methods,” Example I above.

Methylation and statistical analysis. Methylation and statistical analysis was as described herein under Example 1.

Results

CIMP Analysis. It has previously been reported that a subset of colorectal and gastric tumors display a CpG island methylator phenotype (“CIMP”), characterized by widespread, aberrant hypermethylation changes affecting multiple loci in a single tumor (Toyota et al., Proc. Natl. Acad. Sci. USA 96:8681-8686, 1999; Toyota et al., Cancer Res. 59:5438-5442, 1999). This is reflected in a bimodal distribution of the frequency of the number of genes methylated in a group of tumors (Toyota et al., Proc. Natl. Acad. Sci. USA 96:8681-8686, 1999). CIMP tumors are a distinct group of tumors that are defined by a high degree of concordant CpG island hypermethylation of genes exclusively methylated in cancer, or “type-C” genes. CIMP is currently thought to be a new, distinct, yet major pathway of tumorigenesis (Toyota et al., Proc. Natl. Acad. Sci. USA 96:8681-8686, 1999; Toyota et al., Cancer Res. 59:5438-5442, 1999).

Therefore the question of whether esophageal adenocarcinoma tumors exhibit a CpG island methylator phenotype (CIMP) was investigated.

Class A genes of the present invention most closely exemplify the “type-C” genes, because they lack methylation in the normal tissues. The distribution of the number of Class A genes methylated was examined for EAC (FIG. 6).

FIG. 6 shows, according to the present invention, methylation frequency distributions in the progression of esophageal adenocarcinoma. The data was dichotomized at 4 PMR, with 4 PMR and higher designated as methylated, and below 4 PMR as unmethylated. The proportion of patients with zero to three (Class A), zero to nine (Classes A+D) and zero to fourteen CpG islands (Classes A+B+C+D) methylated in each tissue is shown. Class E and F CpG islands were not included since there was no variation in the frequency of methylation between the different tissue. The letter “n” equals the number of samples analyzed in each tissue.

However, the frequency of genes methylated in the adenocarcinoma tissue did not show the expected bimodal distribution of CIMP (FIG. 6) (Toyota et al., Cancer Res. 59:5438-5442, 1999). Similar results were observed when Class D genes, which also exhibit type C methylation, were included along with Class A (FIG. 6, middle panel) and when Classes A, B, C and D genes were combined (FIG. 6, right panel). Classes E and F genes were not included since they did not exhibit any methylation variation between the different tissue types.

There was a single sample with 10 out of 14 Class A-D genes methylated (FIG. 1, Case #3 and FIG. 6). However, this sample only stands out when Class B genes, which are methylated in normal esophageal mucosa and therefore do not satisfy the definition of “type-C” genes that constitute the CIMP phenotype, are included.

Therefore, there was no clear evidence of a separate group of CIMP tumors in the present study of esophageal adenocarcinoma, as has been previously defined for colorectal and gastric cancer.

However CpG island hypermethylation in EAC did occur across multiple loci in a given sample. Furthermore, the number of loci hypermethylated in a single sample increased as the disease progressed through different histological stages (FIG. 6). The bimodal distributions seen in IM tissues (FIG. 6) can be fully attributed to the concurrent association with dysplasia or cancer described herein above.

### EXAMPLE 5

Array- and Microarray-Based Applications

Microarray-based embodiments are within the scope of the present invention. For example, one such array-based embodiment uses differential methylation hybridization (“DMH”), (Huang et al., Hum. Mol. Genet., 8:459-470, 1999; Yan et al., Clin. Cancer Res. 6:1432-38, 2000). DMH is applied to screen paired test and normal samples and to determine whether patterns (see “Epigenetic patterns,” herein under Example 1) of specific epigenetic alterations correlate with pathological parameters in the tissue samples analyzed. “Amplicons” (Id), representing a pool of methylated CpG DNA derived from these samples, are used as hybridization probes in an array panel containing the CpG island tags of the present invention.

Accordingly, one or more of the CpG island sequences associated with 19 of the 20 disclosed gene sequences (i.e., APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS (see TABLES I and II, above; and see under “Definitions,” above), or methylation-altered DNA sequence embodiments thereof, can be used as CpG island tags in an array or microarray-based assay embodiment. These 19 gene sequence regions are defined herein by the oligomeric primers and probes corresponding to SEQ ID NOs: 1-54, 58-60, 64 and 65 (see TABLE II, above; SEQ ID NOs:61-63 correspond to the ACTB “control” gene region used in the present analysis (see EXAMPLE 1, below)). Associated CpG island sequences are (based on the fact that the methylation state of a portion of a given CpG island is generally representative of the island as a whole) those contiguous sequences of genomic DNA that encompass at least one nucleotide of the sequences defined by these specific oligonucleotide primers and probes, and satisfy the criteria of having both a frequency of CpG dinucleotides corresponding to an Observed/Expected Ratio>0.6, and a GC Content>0.5.

These CpG island tags are then arrayed on solid supports (e.g., nylon membranes, silicon, etc.), and probed with amplicons representing a pool of methylated CpG DNA, from test (e.g., tumor) or reference samples. The differences in test and reference signal intensities on screened CpG island arrays reflect methylation alterations of corresponding sequences in the test DNA.

Comparison of the resulting data with the epigenetic patterns disclosed herein allows for a diagnostic or prognostic determination.

Therefore, according to this embodiment, pattern analysis (see working Examples 1-4, below) in a subset of CpG island tags, affixed to a solid support to form an array or microarray, is used to follow progression during various stages of cancer progression (e.g., gastrointestinal and esophageal dysplasia, gastrointestinal and esophageal metaplasia, Barrett's esophagous, and precancerous conditions in normal esophageal squamus mucosa), and can be used to determine histological grades or stages of tumors, such as esophageal adenocarcinoma.

Other array or microarray embodiments of the present invention will be obvious to those of ordinary skill in the relevant art. Such embodiments include, but are not limited to those wherein the specific primers and/or probes for APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS (see TABLES I and II, above; and see under “Defintions,” above), corresponding to SEQ ID NOs:1-54, 58-60, 64 and 65 (see TABLE II, above; SEQ ID NOs:61-63 correspond to the ACTB “control” gene region used in the present analysis (see EXAMPLE 1, above)) are arrayed on solid supports.

DISCUSSION

There is a need in the art for novel and more sensitive methods of cancer detection, chemoprediction and prognostics. There is a need in the art to define novel coordinate patterns of CpG island methylation changes (i.e., novel epigenetic patterns) at multiple loci during progression of a disease, such as cancer. There is a need in the art to determine tumor-type-specific, and patient-specific epigenetic patterns or fingerprints. There is a need in the art to provide biomarkers or probes, such as EAC-specific biomarkers or probes, that can be used in diagnostic and/or prognostic methods for the treatment of cancer. There is a need in the art to determine whether esophageal adenocarcinoma displays a CIMP. There is a need in the art for novel methods for determining the stage of a tumor. The present invention addresses these needs.

A high-throughput, fluorescence-based methylation assay (MethyLight™) was used herein to examine and define novel hypermethylation patterns of 19 CpG islands and one non-CpG island during the progression of esophageal adenocarcinoma (“EAC”). The genes were thereby segregated into six classes of epigenetic patterns in the various tissue types. This is the most comprehensive methylation survey yet performed on a system having so many distinct histological stages of disease progression. Furthermore, the present analysis of abnormal DNA hypermethylation offers a significant advantage over other approaches, such as gene expression analysis, in that it has greater sensitivity in the presence of contaminating normal cells, a common limiting factor.

DNA hypermethylation, as disclosed herein, is an early epigenetic alteration in the multi-step progression of EAC. The premalignant intestinal metaplasia (“IM,” or Barret's esophagus) is already significantly more methylated than the normal tissue (normal squamous mucosa). The present invention, in certain embodiments, provides the novel finding of frequent hypermethylation of five additional genes in this tumor system: MYOD1, MGMT, CALCA, TIMP3, and HIC1.

The methylation observed for MGMT, TIMP3, and HIC1 in normal tissues may be attributed to the particular region of the gene in which we analyzed methylation levels (Stoger et al., Cell. 73:61-71, 1993; Larsen et al., Hum. Mol. Genet. 2:775-80, 1993; Jones, P. A., Trends Genet. 15:34-37, 1999). These three genes were analyzed at CpG islands located at or downstream of the transcription start site (TABLE 2). However, this does not account for the CALCA methylation we observed, because we analyzed the promoter region of this gene. Low levels of CALCA methylation has been previously reported in normal bone marrow samples of AML patients (Melki et al., Cancer Res. 59:3730-3740, 1999), suggesting that this locus may have a higher propensity to be methylated in normal tissues of cancer patients.

It is of particular interest to note that dysplastic tissues are more frequently methylated than stage I tumors for both Class A (p<0.0001) and B (p=0.0174) (FIG. 1). This is similar to the finding of genetic abnormalities (LOH, deletions and mutations) present in Barrett's esophagus with high grade dysplasia but not present in the adjacent invasive EAC (Barrett et al., Nat. Genet. 22:106-109, 1999). Because stage II-IV tumors appear to be methylated at Class A genes at a similar frequency as dysplasia, this suggests that stage I tumors may actually evolve from a different origin than the dysplastic tissue and higher staged tumors, or may diverge after dysplasia independently from stage II-IV tumors during clonal expansion. Alternatively, but less likely, stage I tumors could undergo a transient reversal of hypermethylation. Tumor development in Barrett's esophagus is proposed to evolve clonally through the linear multistep pathway of metaplasia-dysplasia-tumor (Zhuang et al., Cancer Res. 56:1961-4, 1996). However, the occurrence of genetic and, according to the present invention, epigenetic alterations in a non-linear order, indicates that the clonal evolution of EAC is more complex than originally predicted (Barrett et al., Nat. Genet. 22:106-109, 1999). A similar observation has been described for different stages of bladder tumors (Salem et al., Cancer Res. 60:2473-2476, 2000).

There was, under the present analysis, no clear evidence, aside from one tumor with 10 genes methylated, for a separate cluster of tumors with extensive concordant methylation, indicative of a CpG island methylator phenotype (“CIMP”). Similar results were obtained even when only “type-C” genes, as defined for CIMP (methylated in cancer, not methylated in normal tissues; Toyota et al., Proc. Natl. Acad. Sci. USA 96:8681-8686, 1999; Toyota et al., Cancer Res. 59:5438-5442, 1999), were examined. Interestingly, the “type-C” genes in EAC differ from those described for colorectal cancer (Id). For example, ESR1 is classified as a “type-A” (defined as methylated in aging normal tissues) rather than a “type-C” gene in colorectal cancer, because it is frequently methylated in the normal colonic epithelium of aging individuals (Id). However, in esophageal adenocarcinoma, ESRI clearly behaves as a “type-C” gene. This may be attributed to the difference in the technology used to measure hypermethylation, or more likely may be due to differences in tissue types.

According to the present invention, there is a tissue-specific and tumor-specific propensity for particular genes to become hypermethylated. For instance, APC is hypermethylated in normal stomach, but not in normal esophageal mucosa. The tumor-specificity of hypermethylation is illustrated by the lack of detectable methylation of the two Class E genes TGFBR2 and RB1, which are frequently hypermethylated in gastric and lung tumors, and retinoblastoma tumors, respectively (Stirzaker et al., Cancer Res. 57:2229-2237, 1997; Kang et al., Oncogene 18:7280-7286, 1999; Hougaard et al., Br. J. Cancer 79:1005-1011,1999).

The tumor-specificity of CpG island hypermethylation suggests that there may be tissue-specific trans-acting factors that modulate methylation changes of these CpG islands during tumorigenesis and which differ between esophageal adenocarcinomas and other tumor types. Alternatively, there may be a lack of selective advantage to the silencing of these genes in esophageal adenocarcinomas by DNA methylation. There are two scenarios in which this would be the case. One is if the gene in question has been inactivated by a different, genetic mechanism, rendering hypermethylation of no further selective advantage. The other is if the gene does not play a role in tumor suppression in this particular tumor system.

Although alterations in DNA methylation changes are common events in tumorigenesis, the underlying mechanism is unclear. Abnormal methylation, at least in colorectal tumors, is not due to a mere upregulation of the DNA methyltranseferase genes, suggesting that other major players are involved (Eads et al., Cancer Res. 59:2302-2306, 1999). The present invention provides some first glimpses into the process underlying these abnormal methylation changes.

According to the present invention, different, functionally unrelated, genes can behave in distinct classes with respect to their methylation changes within various tissues of EAC progression. The CpG island hypermethylation does not appear to be a random, stochastic process (although there is a stochastic component), but rather a step-wise process that involves multiple, distinct groups of alterations. This is consistent with the existence of several different mechanisms that protect against CpG island hypermethylation. In this scenario, the concerted changes seen at different CpG islands would be the result of the loss of a different type of protective element at different stages of disease progression. This finding does not appear to be dependent on the location of the CpG island relative to the gene, since both promoter and internal CpG islands were observed in all gene classes. The structural features of these CpG islands were also examined under the present analysis by analyzing the % GC content, the observed/expected CpG ratio and the CpG:GpC ratio and found no association with gene class (TABLE 2).

According to the present invention, the IM or NE samples themselves, with or without associated dysplasia or cancer, were histologically indistinguishable, yet molecularly distinct. NE and IM samples derived from individuals with concurrent distally located dysplasia or malignancy show a statistically higher incidence of CpG island hypermethylation. These findings were confirmed herein in the IM tissues in a completely independent study. This provides strong support for the use of epigenetic markers, particularly Class A and B genes, as disease screening tools and as predictive markers for the progression of more advanced staged disease.

The methylation profiles of the present invention provide methods and compositions for the early detection of cancer. Such a molecular diagnostic approach using normal and/or premalignant tissues to identify patients with cancer or at elevated risk for developing cancer provides an opportunity for early intervention. Furthermore, a benefit of using CpG island hypermethylation as a diagnostic or prognostic marker is that it can easily be detected in a field of normal cell contamination as a gain of signal, unlike loss of gene expression (e.g., LOH and deletion analysis), which is difficult to resolve in a sample with contaminating normal cells.

### SUMMARY

According to the present invention, the −19 CpG islands (TABLES I and II) studied segregate into six classes of epigenetic patterns in the various tissue types. Each class undergoes unique epigenetic changes at different steps of disease progression of EAC. The methylation profiles provide methods and compositions for the early detection of cancer.

I claim:

1. A method for diagnosing cancer or cancer-related conditions from tissue samples, comprising:
(a) obtaining a tissue sample from a test tissue or region to be diagnosed;
(b) performing a methylation assay of the tissue sample, wherein the methylation assay determines the methylation state of genomic CpG sequences, wherein the genomic CpG sequences are located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR, and combinations thereof; and
(c) making a diagnostic or prognostic prediction of the cancer based, at least in part, upon the methylation state of the genomic CpG sequences.

2. The method of claim 1, wherein the genomic CpG sequences located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS, correspond to genomic CpG sequences of CpG islands.

3. The method of claim 1, wherein the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR gene sequences are those defined by the specific oligonucleotide primers and probes corresponding to SEQ ID Nos:1-60, 64 and 65, as listed in TABLE II, or portions thereof.

4. The method of claim 2 wherein the CpG islands are located within the promoter regions of one or more of the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2 and TYMS genes.

5. The method of claim 2, wherein the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, and TYMS gene sequences correspond to any CpG island sequences associated with the sequences defined by the specific oligonucleotide primers and probes corresponding to SEQ ID NOs:1-54, 58-60, 64 and 65, as listed in TABLE II, or portions thereof, and wherein the associated CpG island sequences are those contiguous sequences of genomic DNA that encompass at least one nucleotide of the sequences defined by the specific oligonucleotide primers and probes corresponding to SEQ ID NOs:1-54, 58-60, 64 and 65, and satisfy the criteria of having both a frequency of CpG dinucleotides corresponding to an Observed/Expected Ratio>0.6, and a GC Content>0.5.

6. The method of claim 1, wherein the genomic CpG sequences are located within at least one gene sequence selected from the group consisting of APC, CDKN2A, MYOD1, CALCA, ESR1, MGMT and TIMP3, and combinations thereof.

7. The method of claim 6, wherein the genomic CpG sequences located within at least one gene sequence selected from the group consisting of APC, CDKN2A, MYOD1, CALCA, ESR1, MGMT and TIMP3, correspond to genomic CpG sequences of CpG islands.

8. The method of claim 6, wherein the APC, CDKN2A, MYOD1, CALCA, ESR1, MGMT and TIMP3 gene sequences are those defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, respectively, as listed in TABLE II.

9. The method of claim 7 wherein the CpG islands are located within the promoter regions of one or more of the APC, CDKN2A, MYOD1, CALCA, ESR1, MGMT and TIMP3 genes.

10. The method of claim 7 wherein the APC, CDKN2A, MYOD1, CALCA, ESR1, MGMT and TIMP3 gene sequences correspond to any CpG island sequences associated with the sequences defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, respectively, as listed in TABLE II, or portions thereof, and wherein the associated CpG island sequences are those contiguous sequences of genomic DNA that encompass at least one nucleotide of the sequences defined by the specific oligonucleotide primers and probes corresponding to SEQ ID NOs:19-21, SEQ ID NOs:1-3, SEQ ID NOs:7-9, SEQ ID NOs:10-12, SEQ ID NOs:4-6, SEQ ID NOs:16-18 and SEQ ID NOs:13-15, and satisfy the criteria of having both a frequency of CpG dinucleotides corresponding to an Observed/Expected Ratio>0.6, and a GC Content>0.5.

11. The method of claim 1, wherein the cancer or cancer-related condition is selected from the group consisting of gastrointestinal or esophageal adenocarcinoma, gastrointestinal or esophageal dysplasia, gastrointestinal or esophageal metaplasia, Barrett's intestinal tissue, pre-cancerous conditions in normal esophageal squamous mucosa, and combinations thereof.

12. The method of claim 11, wherein the cancer is esophageal adenocarcinoma, and wherein making a diagnostic or prognostic prediction of the cancer, based upon the methylation state of the genomic CpG sequences provides for classification of the adenocarcinoma by grade or stage.

13. The method of claim 6, wherein the cancer or cancer-related condition is selected from the group consisting of gastrointestinal or esophageal adenocarcinoma, gastrointestinal or esophageal dysplasia, gastrointestinal or esophageal metaplasia, Barrett's intestinal tissue, pre-cancerous conditions in normal esophageal squamous mucosa, and combinations thereof.

14. The method of claim 13, wherein the cancer is esophageal adenocarcinoma, and wherein making a diagnostic or prognostic prediction of the cancer, based upon the methylation state of the genomic CpG sequences provides for classification of the adenocarcinoma by grade or stage.

15. The method of claim 1, wherein the methylation assay used to determine the methylation state of genomic CpG sequences is selected from the group consisting of MethylLight™, MS-SNuPE, MSP, COBRA, MCA, and DMH, and combinations thereof.

16. The method of claim 6, wherein the methylation assay used to determine the methylation state of genomic CpG sequences is selected from the group consisting of MethylLight™, MS-SNuPE, MSP, COBRA, MCA and DMH, and combinations thereof.

17. The method of claim 1, wherein the methylation assay used to determine the methylation state of genomic CpG sequences is based, at least in part, on an array or microarray comprising CpG-containing sequences located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR.

18. The method of claim 17, wherein the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, and TYMS gene sequences correspond to any CpG island sequences associated with the sequences defined by the specific oligonucleotide primers and probes corresponding to SEQ ID NOs:1-54, 58-60, 64 and 65, as listed in TABLE II, or portions thereof, and wherein the associated CpG island sequences are those contiguous sequences of genomic DNA that encompass at least one nucleotide of the sequences defined by the specific oligonucleotide primers and probes corresponding to SEQ ID NOs:1-54, 58-60, 64 and 65, and satisfy the criteria of having both a frequency of CpG dinucleotides corresponding to an Observed/Expected Ratio>0.6, and a GC Content>0.5.

19. The method of claim 17, wherein the APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR gene sequences are those defined by, or correspond to the specific oligonucleotide primers and probes corresponding to SEQ ID NOs:1-60, 64 and 65, as listed in TABLE II, or portions thereof.

20. The method of claim 1 wherein the methylation state of genomic CpG sequences that is determined is that of hypermethylation, hypomethylation or normal methylation.

21. A kit useful for diagnosis or prognosis of cancer or cancer-related conditions, comprising a carrier means containing one or more containers comprising:
(a) a container containing a probe or primer which hybridizes to any region of a sequence located within at least one gene sequence selected from the group consisting of APC, ARF, CALCA, CDH1, CDKN2A, CDKN2B, ESR1, GSTP1, HIC1, MGMT, MLH1, MYOD1, RB1, TGFBR2, THBS1, TIMP3, CTNNB1, PTGS2, TYMS and MTHFR; and
(b) additional standard methylation assay reagents required to affect detection of methylated CpG-containing nucleic acid based, at least in part, on the probe or primer.

22. The kit of claim 21, wherein the additional standard methylation assay reagents are standard reagents for performing a methylation assay from the group consisting of MethyLight™, MS-SNuPE, MSP, COBRA, MCA and DMH, and combinations thereof.

23. The kit of claim 21, wherein the probe or primer comprises at least about 12 to 15 nucleotides of a sequence selected from the group consisting of SEQ ID NOs:1-60, 64 and 65, as listed in TABLE II.

24. A kit useful for diagnosis or prognosis of cancer or cancer-related conditions, comprising a carrier means containing one or more containers comprising:
(a) an array or micorarray comprising sequences of at least about 12 to 15 nucleotides of a sequence selected from the group consisting of SEQ ID NOs:1-60, 64, 65, and any sequence located within a CpG island sequence associated with SEQ ID NOs:1-54, 58-60, 64 and 65.


SEQUENCE LISTING (65 sequences)

SEQ ID NO: 1 - DNA, 22 bp, Homo sapiens
tggaattttcggttgattggtt

SEQ ID NO: 2 - DNA, 19 bp, Homo sapiens
aacaacgtccgcacctcct

SEQ ID NO: 3 - DNA, 18 bp, Homo sapiens
acccgaccccgaaccgcg

SEQ ID NO: 4 - DNA, 19 bp, Homo sapiens
ggcgttcgttttgggattg

SEQ ID NO: 5 - DNA, 19 bp, Homo sapiens
gccgacacgcgaactctaa

SEQ ID NO: 6 - DNA, 24 bp, Homo sapiens
cgataaaaccgaacgacccgacga

SEQ ID NO: 7 - DNA, 18 bp, Homo sapiens
gagcgcgcgtagttagcg

SEQ ID NO: 8 - DNA, 17 bp, Homo sapiens
tccgacacgccctttcc

SEQ ID NO: 9 - DNA, 30 bp, Homo sapiens
ctccaacacccgactactatatccgcgaaa

SEQ ID NO: 10 - DNA, 23 bp, Homo sapiens
gttttggaagtatgagggtgacg

SEQ ID NO: 11 - DNA, 19 bp, Homo sapiens
ttcccgccgctataaatcg

SEQ ID NO: 12 - DNA, 30 bp, Homo sapiens
attccgccaatacacaacaaccaataaacg

SEQ ID NO: 13 - DNA, 21 bp, Homo sapiens
gcgtcggaggttaaggttgtt

SEQ ID NO: 14 - DNA, 22 bp, Homo sapiens
ctctccaaaattaccgtacgcg

SEQ ID NO: 15 - DNA, 19 bp, Homo sapiens
aactcgctcgcccgccgaa

SEQ ID NO: 16 - DNA, 28 bp, Homo sapiens
ctaacgtataacgaaaatcgtaacaacc

SEQ ID NO: 17 - DNA, 25 bp, Homo sapiens
agtatgaagggtaggaagaattcgg

SEQ ID NO: 18 - DNA, 30 bp, Homo sapiens
ccttacctctaaataccaaccccaaacccg

SEQ ID NO: 19 - DNA, 19 bp, Homo sapiens
gaaccaaaacgctccccat

SEQ ID NO: 20 - DNA, 27 bp, Homo sapiens
ttatatgtcggttacgtgcgtttatat

SEQ ID NO: 21 - DNA, 22 bp, Homo sapiens
cccgtcgaaaacccgccgatta

SEQ ID NO: 22 - DNA, 19 bp, Homo sapiens
acgggcgttttcggtagtt

SEQ ID NO: 23 - DNA, 20 bp, Homo sapiens
ccgaacctccaaaatctcga

SEQ ID NO: 24 - DNA, 26 bp, Homo sapiens
cgactctaaaccctacgcacgcgaaa

SEQ ID NO: 25 - DNA, 26 bp, Homo sapiens
aattttaggttagagggttatcgcgt

SEQ ID NO: 26 - DNA, 22 bp, Homo sapiens
tccccaaaacgaaactaacgac

SEQ ID NO: 27 - DNA, 19 bp, Homo sapiens
cgcccacccgacctcgcat

SEQ ID NO: 28 - DNA, 20 bp, Homo sapiens
aggaaggagagagtgcgtcg

SEQ ID NO: 29 - DNA, 21 bp, Homo sapiens
cgaataatccaccgttaaccg

SEQ ID NO: 30 - DNA, 29 bp, Homo sapiens
ttaacgacactcttcccttctttcccacg

SEQ ID NO: 31 - DNA, 23 bp, Homo sapiens
gtcggcgtcgtgatttagtattg

SEQ ID NO: 32 - DNA, 23 bp, Homo sapiens
aaactacgacgacgaaactccaa

SEQ ID NO: 33 - DNA, 29 bp, Homo sapiens
aaacctcgcgacctccgaaccttataaaa

SEQ ID NO: 34 - DNA, 18 bp, Homo sapiens
ctatcgccgcctcatcgt

SEQ ID NO: 35 - DNA, 30 bp, Homo sapiens
cgttatatatcgttcgtagtattcgtgttt

SEQ ID NO: 36 - DNA, 22 bp, Homo sapiens
cgcgacgtcaaacgccactacg

SEQ ID NO: 37 - DNA, 19 bp, Homo sapiens
cggaagcgttcgggtaaag

SEQ ID NO: 38 - DNA, 18 bp, Homo sapiens
aattccaccgccccaaac

SEQ ID NO: 39 - DNA, 29 bp, Homo sapiens
tttccgccaaatatcttttcttcttcgca

SEQ ID NO: 40 - DNA, 18 bp, Homo sapiens
cgacgcaccaacctaccg

SEQ ID NO: 41 - DNA, 25 bp, Homo sapiens
gttttgagttggttttacgttcgtt

SEQ ID NO: 42 - DNA, 19 bp, Homo sapiens
acgccgcgctcacctccct

SEQ ID NO: 43 - DNA, 17 bp, Homo sapiens
ggaaaggcgcgtcgagt

SEQ ID NO: 44 - DNA, 18 bp, Homo sapiens
tcccctatcccaaacccg

SEQ ID NO: 45 - DNA, 18 bp, Homo sapiens
cgcgcgtttcccgaaccg

SEQ ID NO: 46 - DNA, 22 bp, Homo sapiens
ttagttcgcgtatcgattagcg

SEQ ID NO: 47 - DNA, 18 bp, Homo sapiens
actaaacgccgcgtccaa

SEQ ID NO: 48 - DNA, 21 bp, Homo sapiens
tcacgtccgcgaaactcccga

SEQ ID NO: 49 - DNA, 18 bp, Homo sapiens
gcgcggagcgtagttagg

SEQ ID NO: 50 - DNA, 20 bp, Homo sapiens
caaaccccgctactcgtcat

SEQ ID NO: 51 - DNA, 21 bp, Homo sapiens
cacgaacgacgccttcccgaa

SEQ ID NO: 52 - DNA, 19 bp, Homo sapiens
cggcgttaggaaggacgat

SEQ ID NO: 53 - DNA, 24 bp, Homo sapiens
tctcaaactataacgcgcctacat

SEQ ID NO: 54 - DNA, 29 bp, Homo sapiens
ccgaataccgacaaaataccgatacccgt

SEQ ID NO: 55 - DNA, 29 bp, Homo sapiens
tggtagtgagagttttaaagatagttcga

SEQ ID NO: 56 - DNA, 18 bp, Homo sapiens
cgcctcatcttctcccga

SEQ ID NO: 57 - DNA, 27 bp, Homo sapiens
tctcataccgctcaaaatccaaacccg

SEQ ID NO: 58 - DNA, 19 bp, Homo sapiens
gttaggcggttagggcgtc

SEQ ID NO: 59 - DNA, 19 bp, Homo sapiens
ccgaacgcctccatcgtat

SEQ ID NO: 60 - DNA, 31 bp, Homo sapiens
caacatcgtctacccaacacactctcctacg

SEQ ID NO: 61 - DNA, 25 bp, Homo sapiens
tggtgatggaggaggtttagtaagt

SEQ ID NO: 62 - DNA, 27 bp, Homo sapiens
aaccaataaaacctactcctcccttaa

SEQ ID NO: 63 - DNA, 30 bp, Homo sapiens
accaccacccaacacacaataacaaacaca

SEQ ID NO: 64 - DNA, 22 bp, Homo sapiens
tggagttttcggttgattggtt

SEQ ID NO: 65 - DNA, 19 bp, Homo sapiens
aacaacgcccgcacctcct